(19) United States
(12) Patent Application Publication
(10) Pub. No.: US 2026/0253688 A1
(43) Pub. Date: Aug. 27, 2026

(54) SYSTEM

(71) Applicant: SoftBank Group Corp., Tokyo (JP)

(72) Inventor: Mai SAKAI, Tokyo (JP)

(73) Assignee: SoftBank Group Corp., Tokyo (JP)

(21) Appl. No.: 19/534,769

(22) Filed: Feb. 10, 2026

(30) Foreign Application Priority Data
Feb. 21, 2025 (JP) 2025-027062

(51) Int. Cl. G16H 10/60; G06Q 10/10; G16H 40/67

(52) U.S. Cl. CPC G16H 10/60; G06Q 10/10; G16H 40/67

(57) ABSTRACT

The system according to the embodiment comprises a reception unit, an analysis unit, a determination unit, a request unit, and a provisional request unit. The reception unit receives information from a user. The analysis unit analyzes the information received by the reception unit. The determination unit determines the necessity of medical consultation based on the information analyzed by the analysis unit. The request unit requests an ambulance when the determination unit determines that medical consultation is necessary. The provisional request unit makes a provisional request to a hospital for patient transport when the request unit has requested an ambulance.

### CROSS-REFERENCE TO RELATED APPLICATION

The present application claims priority to and incorporates by reference the entire contents of Japanese Patent Application No. 2025-027062 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to promptly and accurately determine the necessity of medical consultation for an illness or injury and to take appropriate action.

### SUMMARY OF THE INVENTION

The system according to the embodiment comprises a reception unit, an analysis unit, a determination unit, a request unit, and a provisional request unit. The reception unit receives information from a user. The analysis unit analyzes the information received by the reception unit. The determination unit determines the necessity of medical consultation based on the information analyzed by the analysis unit. The request unit requests an ambulance when the determination unit determines that medical consultation is necessary. The provisional request unit makes a provisional request to a hospital for patient transport when the request unit has requested an ambulance.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

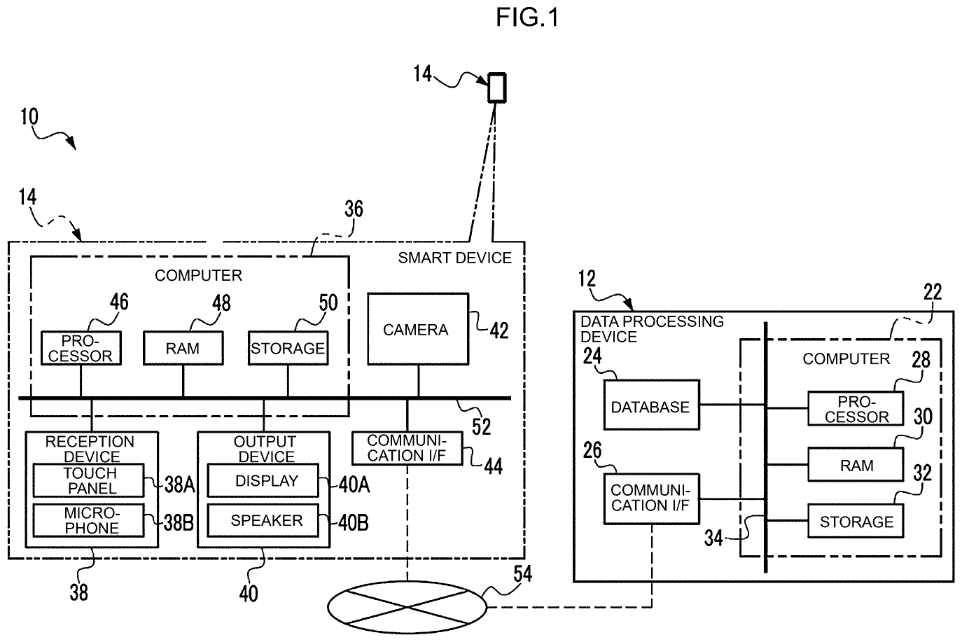

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

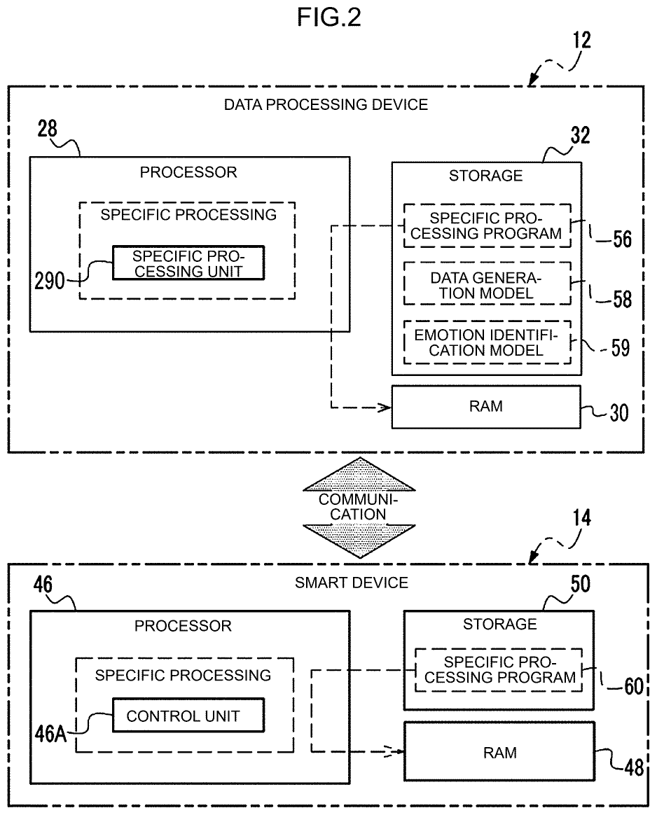

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that uses AI to determine illnesses or injuries of humans or pets from photographs or situations and judges whether medical consultation is necessary. In this system, the user inputs photographs or situations of illnesses or injuries, and the AI analyzes the information to determine the necessity of medical consultation. If medical consultation is necessary, the AI determines whether a request for an ambulance is required and, if necessary, coordinates with the ambulance. Furthermore, when coordinating with the ambulance, the AI also makes a provisional request to the hospital for patient transport, thereby supporting the shortening of transport time by preparing the hospital for acceptance. For example, the user inputs photographs or situations of illnesses or injuries. For instance, if a pet is injured, the user takes a photograph of the injury and inputs the situation in text. This information is input to the AI. Next, the AI analyzes the input information. Based on the photograph and text information, the AI determines the condition of the illness or injury. For example, the AI analyzes the degree of bleeding and the depth of the wound from the photograph and understands the details of the symptoms from the text. After determining the condition of the illness or injury, the AI judges the necessity of medical consultation. For example, if the injury is minor, the AI recommends first aid at home, and if the injury is severe, it recommends medical consultation. If medical consultation is necessary, the AI determines whether a request for an ambulance is required. For example, if there is a life-threatening situation or urgent treatment is needed, the AI recommends requesting an ambulance. If a request for an ambulance is necessary, the AI coordinates with the ambulance. Specifically, based on the user's location information, the AI arranges the nearest ambulance and provides necessary information until the ambulance arrives. Furthermore, when coordinating with the ambulance, the AI also makes a provisional request to the hospital for patient transport. This allows the hospital to prepare for acceptance and shortens the transport time. For example, the AI notifies the hospital of the patient's condition and expected arrival time and prompts the preparation of necessary medical staff and equipment. With this mechanism, early detection and rapid response to illnesses or injuries are possible, improving patient safety and the quality of treatment. Thus, the AI-based system enables early detection and rapid response to illnesses or injuries, improving patient safety and treatment quality. Specifically, the system comprises multiple hardware and software elements such as an image input unit, text input unit, voice input unit, multimodal information integration unit, image analysis neural network (e.g., convolutional neural network), natural language processing model (e.g., Transformer-based large language model), voice recognition model, state estimation algorithm, severity determination module, ambulance request determination module, hospital provisional request module, transport destination optimization module, and information notification interface. The system accepts image data (e.g., RGB images, resolution 1280×720 pixels, JPEG format) taken by the user with a smartphone or tablet, text data (e.g., symptom description “bleeding from the right foreleg,” “labored breathing”), and voice data (e.g., WAV format, 16 kHz sampling, user's spoken symptom description). The image data undergoes preprocessing such as noise removal, resizing, and normalization, and is converted into tensor format (e.g., 3×224×224). Text data is tokenized and vectorized, and voice data undergoes spectrogram conversion and acoustic feature extraction (e.g., MFCC). These multimodal data are input into a multimodal integration model (e.g., image-text integrated Transformer). The image analysis neural network performs segmentation of bleeding areas, estimation of wound depth (e.g., pixel-level depth map generation), and detection of abnormal sites (e.g., object detection algorithms such as YOLO). The natural language processing model extracts keywords from symptom descriptions (e.g., “bleeding,” “swelling,” “dyspnea”), understands the time-series progression of symptoms, and performs severity scoring (e.g., probability values from 0.0 to 1.0). The voice recognition model converts voice to text, which is similarly input to the NLP model. The state estimation algorithm integrates features obtained from image, text, and voice and inputs them to the severity determination module. The severity determination module uses, for example, multilayer perceptrons or decision trees to output label classification such as “mild,” “moderate,” “severe,” and severity scores (e.g., 0.15, 0.72). The ambulance request determination module sets the request flag ON when the severity score is 0.7 or higher or when specific keywords such as “consciousness disorder” or “massive bleeding” are extracted. When the request flag is ON, the transport destination optimization module searches for the nearest ambulance and an available hospital based on the user's location information (GPS coordinates, e.g., latitude 35.6, longitude 139.7). The hospital provisional request module automatically sends structured data such as patient condition (e.g., severity 0.85, vital signs “heart rate 120, blood pressure 90/60”), expected arrival time (e.g., current time+15 minutes), and necessary medical resources (e.g., surgeon, transfusion equipment) to the hospital system. This enables the hospital to automate acceptance preparation, shorten transport time, and accelerate the start of treatment. For AI model training, large-scale datasets of past case images, symptom descriptions, diagnostic results, and transport records are used, and optimization is performed using loss functions such as cross-entropy and MSE. The model is trained in a distributed manner on GPU clusters, and inference is performed at high speed on edge devices or cloud servers. Examples of AI output include “Image input: bleeding image of right foreleg, text input: ‘labored breathing’→output: severity 0.82, ambulance request required” and “Image input: small cut image, text input: ‘little pain’→output: severity 0.18, first aid recommended.” In subsequent processing, the severity score and request flag are input to the ambulance arrangement API and hospital coordination API, and automated processing such as information notification, transport arrangement, and medical resource allocation is performed. As a technical effect, the system eliminates subjective human judgment and manual information transmission, realizes high-precision and rapid state determination and transport arrangement by AI, greatly reduces misjudgment and delays, improves patient survival rates, reduces the burden on medical sites, and streamlines the entire transport process. Specific application fields include emergency response in general households, triage in animal hospitals, emergency medical support in remote areas and elderly facilities, and response to multiple casualties during disasters. Unlike conventional human visual inspection, telephone contact, and manual judgment, the present invention uses unconventional and technical methods such as AI inference in high-dimensional feature space, a combination of rule-based and machine learning, and real-time data coordination, thereby contributing to the improvement of computer technology itself.

The system according to the embodiment comprises a reception unit, an analysis unit, a determination unit, a request unit, and a provisional request unit. The reception unit receives information from the user. The information from the user may include, for example, photographs of illnesses or injuries, symptom descriptions, location information, and the like, but is not limited thereto. The reception unit, for example, receives photographs of illnesses or injuries taken by the user with a smartphone. The reception unit can also receive text information or voice information input by the user. For example, the reception unit receives symptom descriptions input by the user in text format. Furthermore, the reception unit can acquire the user's location information and use it for arranging an ambulance. The analysis unit analyzes the information received by the reception unit. The analysis unit, for example, uses image analysis algorithms to analyze photographs of illnesses or injuries. For example, the analysis unit uses image analysis algorithms to analyze the degree of bleeding and the depth of wounds from photographs of illnesses or injuries. The analysis unit can also use natural language processing technology to analyze text information. For example, the analysis unit uses natural language processing technology to determine the condition of the illness or injury from symptom descriptions input by the user. Furthermore, the analysis unit can use voice analysis technology to analyze voice information. For example, the analysis unit uses voice analysis technology to analyze symptom descriptions spoken by the user. The determination unit determines the necessity of medical consultation based on the information analyzed by the analysis unit. The determination unit, for example, evaluates the severity and urgency of the condition of the illness or injury based on the analysis result. For example, the determination unit determines the necessity of medical consultation based on the degree of bleeding, depth of wounds, and details of symptoms. The request unit requests an ambulance when it is determined that medical consultation is necessary. The request unit, for example, arranges the nearest ambulance based on the user's location information. For example, the request unit requests an ambulance using an emergency call system. The provisional request unit makes a provisional request to the hospital for patient transport when an ambulance has been requested. The provisional request unit, for example, notifies the hospital of the patient's condition and expected arrival time. For example, the provisional request unit provides the hospital with detailed information about the patient's symptoms, vital signs, expected arrival time, and the like. This allows the hospital to prepare for acceptance and shortens the transport time. Thus, the system according to the embodiment can efficiently receive and analyze user information, determine the necessity of medical consultation, and, if necessary, request an ambulance and make a provisional request to the hospital. Specifically, the system comprises, as the reception unit, an image input module, a text input module, a voice input module, and a location information acquisition module, which receive image data (e.g., JPEG format, 1280×720 pixels), text data (e.g., “bleeding from the right foreleg”), voice data (e.g., WAV format, 16 kHz), and GPS coordinates (e.g., latitude 35.6, longitude 139.7) sent from the user terminal. The reception unit performs preprocessing such as noise removal, resizing, and normalization on image data and converts it into tensor format (e.g., 3×224×224). Text data is tokenized and vectorized, and voice data undergoes spectrogram conversion and MFCC extraction. The analysis unit uses an image analysis neural network (e.g., convolutional neural network) to perform segmentation of bleeding areas and estimation of wound depth from images, and uses a natural language processing model (e.g., Transformer-based large language model) to extract symptom keywords and perform severity scoring from text. Voice information is converted to text by a voice recognition model and input to the NLP model. The analysis unit integrates these multimodal features and inputs them to a state estimation algorithm (e.g., multilayer perceptron). The determination unit judges the necessity of medical consultation based on the output of the state estimation algorithm (e.g., severity score 0.0-1.0, labels such as “mild,” “moderate,” “severe”) using threshold judgment or rule-based logic. The request unit sets the request flag ON when the severity score is 0.7 or higher or when specific keywords (e.g., “consciousness disorder”) are extracted, and arranges the nearest ambulance based on the user's location information. The request unit cooperates with the ambulance arrangement API and emergency call system to automatically send necessary information (e.g., patient condition, location information). The provisional request unit activates the hospital provisional request module when an ambulance is requested and sends structured data such as patient condition (e.g., severity 0.85, vital signs “heart rate 120, blood pressure 90/60”), expected arrival time (e.g., current time+15 minutes), and necessary medical resources (e.g., surgeon, transfusion equipment) to the hospital system. This enables the hospital to automate acceptance preparation, shorten transport time, and accelerate the start of treatment. For AI model training, large-scale datasets of past case images, symptom descriptions, diagnostic results, and transport records are used, and optimization is performed using loss functions such as cross-entropy and MSE. The model is trained in a distributed manner on GPU clusters, and inference is performed at high speed on edge devices or cloud servers. Examples of AI output include “Image input: bleeding image of right foreleg, text input: ‘labored breathing’→output: severity 0.82, ambulance request required” and “Image input: small cut image, text input: ‘little pain’→output: severity 0.18, first aid recommended.” In subsequent processing, the severity score and request flag are input to the ambulance arrangement API and hospital coordination API, and automated processing such as information notification, transport arrangement, and medical resource allocation is performed. As a technical effect, the system eliminates subjective human judgment and manual information transmission, realizes high-precision and rapid state determination and transport arrangement by AI, greatly reduces misjudgment and delays, improves patient survival rates, reduces the burden on medical sites, and streamlines the entire transport process. Specific application fields include emergency response in general households, triage in animal hospitals, emergency medical support in remote areas and elderly facilities, and response to multiple casualties during disasters. Unlike conventional human visual inspection, telephone contact, and manual judgment, the present invention uses unconventional and technical methods such as AI inference in high-dimensional feature space, a combination of rule-based and machine learning, and real-time data coordination, thereby contributing to the improvement of computer technology itself.

The reception unit can receive photographs or situations of illnesses or injuries input by the user. For example, the reception unit receives photographs of illnesses or injuries taken by the user with a smartphone. For instance, the reception unit receives photographs of wounds or swelling taken by the user. The reception unit can also receive text information input by the user. For example, the reception unit receives symptom descriptions or injury situations input by the user in text format. Furthermore, the reception unit can receive voice information input by the user. For example, the reception unit receives symptom descriptions spoken by the user in voice format. By receiving photographs or situations of illnesses or injuries input by the user, the reception unit can obtain accurate information. Specifically, the reception unit comprises multiple hardware and software elements such as an image input module, text input module, voice input module, and location information acquisition module. The image input module receives image data (e.g., JPEG format, 1280×720 pixels, RGB images) sent from the user terminal, performs preprocessing such as noise removal, resizing, and normalization, and converts it into tensor format (e.g., 3×224×224). The text input module tokenizes and vectorizes symptom descriptions or injury situations input by the user (e.g., “bleeding from the right foreleg,” “swelling present”) and converts them into a format suitable for input to the natural language processing model. The voice input module receives voice data spoken by the user (e.g., WAV format, 16 kHz sampling), performs spectrogram conversion and acoustic feature extraction such as MFCC. The location information acquisition module uses the GPS function of the user terminal to acquire latitude and longitude information (e.g., latitude 35.6, longitude 139.7) and uses it for ambulance arrangement and transport destination optimization processing. The reception unit integrates these multimodal data and sends them to the subsequent analysis unit. Examples of input to the AI include “Image: bleeding image of right foreleg, text: ‘labored breathing,’ voice: ‘severe pain’” and “Image: swelling photograph, text: ‘swelling is spreading.’” By accurately and rapidly receiving such diverse information, the reception unit greatly improves the accuracy and reliability of subsequent AI analysis processing and reduces the risk of misjudgment due to erroneous input or missing information. As a technical effect, the reception unit eliminates ambiguity and delays in information collection by manual human work and realizes high-precision and rapid data acquisition by computer, thereby contributing to the efficiency and reliability of the entire medical support process. Specific application fields include emergency response in general households, triage in animal hospitals, emergency medical support in remote areas and elderly facilities, and response to multiple casualties during disasters. Unlike conventional human visual inspection and manual input, the reception unit uses unconventional and technical methods such as data integration in high-dimensional feature space and real-time data coordination, thereby contributing to the improvement of computer technology itself.

The analysis unit can analyze the received photographs or situations and determine the condition of the illness or injury. For example, the analysis unit uses image analysis algorithms to analyze photographs of illnesses or injuries. For instance, the analysis unit uses image analysis algorithms to analyze the degree of bleeding and the depth of wounds from photographs of illnesses or injuries. The analysis unit can also use natural language processing technology to analyze text information. For example, the analysis unit uses natural language processing technology to determine the condition of the illness or injury from symptom descriptions input by the user. Furthermore, the analysis unit can use voice analysis technology to analyze voice information. For example, the analysis unit uses voice analysis technology to analyze symptom descriptions spoken by the user. By analyzing the received photographs or situations, the analysis unit can accurately determine the condition of the illness or injury. Specifically, the analysis unit uses an image analysis neural network (e.g., convolutional neural network) to perform segmentation of bleeding areas and estimation of wound depth (e.g., pixel-level depth map generation), and detection of abnormal sites (e.g., object detection algorithms such as YOLO) from images. The natural language processing model (e.g., Transformer-based large language model) extracts keywords from symptom descriptions (e.g., “bleeding,” “swelling,” “dyspnea”), understands the time-series progression of symptoms, and performs severity scoring (e.g., probability values from 0.0 to 1.0). The voice recognition model converts voice to text, which is similarly input to the NLP model. The analysis unit integrates these multimodal features and inputs them to a state estimation algorithm (e.g., multilayer perceptron or decision tree), thereby outputting the condition of the illness or injury as labels such as “mild,” “moderate,” “severe,” or as severity scores. Examples of input to the AI include “Image: bleeding image, text: ‘labored breathing,’ voice: ‘severe pain’” and “Image: swelling photograph, text: ‘swelling is spreading.’” Examples of output include “severity 0.82, ambulance request required” and “severity 0.18, first aid recommended.” The analysis unit uses trained model weights and parameters to perform feature extraction and inference in high-dimensional space on input data, applying unconventional algorithms different from conventional human visual inspection or simple rule-based processing. As a technical effect, the analysis unit eliminates dependence on subjective human judgment and empirical rules, realizes high-precision and rapid state determination by AI, greatly reduces misjudgment and delays, improves patient survival rates, reduces the burden on medical sites, and streamlines the entire transport process. Specific application fields include emergency response in general households, triage in animal hospitals, emergency medical support in remote areas and elderly facilities, and response to multiple casualties during disasters.

The determination unit can determine the necessity of medical consultation based on the analysis result. For example, the determination unit evaluates the severity and urgency of the condition of the illness or injury based on the analysis result. For instance, the determination unit determines the necessity of medical consultation based on the degree of bleeding, depth of wounds, and details of symptoms. By determining the necessity of medical consultation based on the analysis result, the determination unit enables appropriate response. Specifically, the determination unit uses a state estimation algorithm (e.g., multilayer perceptron, decision tree, rule-based determination logic) to input the severity score (e.g., 0.0-1.0), labels (e.g., “mild,” “moderate,” “severe”), and extracted keywords (e.g., “consciousness disorder,” “massive bleeding”) received from the analysis unit, and performs threshold judgment and branching processing based on combinations of multiple conditions. For example, if the severity score is 0.7 or higher or specific keywords are extracted, the determination unit judges “medical consultation recommended” or “ambulance request,” and if the score is less than 0.3, it judges “first aid at home recommended.” Examples of AI output include “severity 0.82, ambulance request required” and “severity 0.18, first aid recommended,” and the determination unit sends these outputs to the subsequent request unit or notification interface. By using trained determination models or rule sets, the determination unit realizes objective and highly reproducible judgment, unlike conventional subjective or experiential human judgment. As a technical effect, the determination unit reduces misjudgment and delays, contributing to improved patient safety, reduced burden on medical sites, and efficiency of the overall process. Specific application fields include emergency response in general households, triage in animal hospitals, emergency medical support in remote areas and elderly facilities, and response to multiple casualties during disasters.

The request unit can request an ambulance when it is determined that medical consultation is necessary. For example, the request unit arranges the nearest ambulance based on the user's location information. For instance, the request unit requests an ambulance using an emergency call system. By doing so, the request unit can promptly request an ambulance when medical consultation is determined to be necessary. Specifically, the request unit receives the request flag, severity score, and user's location information (GPS coordinates, e.g., latitude 35.6, longitude 139.7) from the determination unit as input, and automatically searches for and arranges the nearest ambulance in cooperation with the ambulance arrangement API and emergency call system. The request unit uses the transport destination optimization module to search for ambulances and hospitals that can arrive via the shortest route from the current location and automatically sends necessary information (e.g., patient condition, location information, severity score) as structured data. Examples of AI output include “request flag ON, transport destination hospital A, ambulance ID123,” and the request unit notifies such information in real time. Unlike conventional human telephone contact and manual arrangement, the request unit performs automated and optimized processing by AI, greatly reducing arrangement delays and information transmission errors. As a technical effect, the request unit realizes rapid and accurate ambulance arrangement, contributing to improved patient survival rates, reduced burden on medical sites, and efficiency of the entire transport process. Specific application fields include emergency response in general households, triage in animal hospitals, emergency medical support in remote areas and elderly facilities, and response to multiple casualties during disasters.

The provisional request unit can make a provisional request to the hospital for patient transport when an ambulance has been requested. For example, the provisional request unit notifies the hospital of the patient's condition and expected arrival time. For instance, the provisional request unit provides the hospital with detailed information about the patient's symptoms, vital signs, expected arrival time, and the like. By making a provisional request to the hospital for patient transport when an ambulance has been requested, the provisional request unit can shorten the transport time. Specifically, the provisional request unit receives ambulance request information from the request unit, patient condition (e.g., severity 0.85, vital signs “heart rate 120, blood pressure 90/60”), expected arrival time (e.g., current time+15 minutes), and necessary medical resources (e.g., surgeon, transfusion equipment) as input, and activates the hospital provisional request module. The provisional request unit automatically sends these information as structured data to the hospital system, enabling the hospital to automate acceptance preparation. Examples of AI output include “patient condition: severity 0.85, vital signs: heart rate 120, blood pressure 90/60, expected arrival time: in 15 minutes, necessary resources: surgeon, transfusion equipment,” and the provisional request unit notifies such information in real time. Unlike conventional human telephone contact and manual information transmission, the provisional request unit performs automated and optimized processing by AI, greatly reducing information transmission delays and treatment delays due to insufficient preparation. As a technical effect, the provisional request unit realizes rapid and accurate hospital acceptance preparation, contributing to shortened transport time, accelerated start of treatment, and improved patient survival rates. Specific application fields include emergency response in general households, triage in animal hospitals, emergency medical support in remote areas and elderly facilities, and response to multiple casualties during disasters.

The provisional request unit can notify the hospital of the patient's condition or expected arrival time and prompt preparation of necessary medical staff or equipment. For example, the provisional request unit notifies the hospital of the patient's condition and expected arrival time. For instance, the provisional request unit provides the hospital with detailed information about the patient's symptoms, vital signs, expected arrival time, and the like. By notifying the hospital of the patient's condition and expected arrival time, the provisional request unit can prompt preparation of necessary medical staff and equipment, enabling rapid response. Specifically, the provisional request unit receives ambulance request information from the request unit, patient condition (e.g., severity 0.85, vital signs “heart rate 120, blood pressure 90/60”), expected arrival time (e.g., current time+15 minutes), and necessary medical resources (e.g., surgeon, transfusion equipment) as input, and activates the hospital provisional request module. The provisional request unit automatically sends these information as structured data to the hospital system, enabling the hospital to automate acceptance preparation. Examples of AI output include “patient condition: severity 0.85, vital signs: heart rate 120, blood pressure 90/60, expected arrival time: in 15 minutes, necessary resources: surgeon, transfusion equipment,” and the provisional request unit notifies such information in real time. Unlike conventional human telephone contact and manual information transmission, the provisional request unit performs automated and optimized processing by AI, greatly reducing information transmission delays and treatment delays due to insufficient preparation. As a technical effect, the provisional request unit realizes rapid and accurate hospital acceptance preparation, contributing to shortened transport time, accelerated start of treatment, and improved patient survival rates. Specific application fields include emergency response in general households, triage in animal hospitals, emergency medical support in remote areas and elderly facilities, and response to multiple casualties during disasters.

The reception unit can estimate the user's emotion and adjust the display method of the input interface based on the estimated emotion. For example, if the user feels anxious, the reception unit provides an interface with calm colors to give a sense of security. If the user is in a hurry, the reception unit provides a simple and intuitive interface to prompt rapid input. Furthermore, if the user is relaxed, the reception unit can provide detailed input options and propose customizable input methods. By adjusting the display method of the input interface according to the user's emotion, the reception unit can reduce the user's stress and facilitate smooth input operations. Emotion estimation is realized by using, for example, an emotion engine or generative AI with emotion estimation functions. Generative AI may be, for example, a text generation AI (such as an LLM) or a multimodal generative AI, but is not limited thereto. Specifically, the reception unit comprises multiple hardware and software elements such as an image input module, text input module, voice input module, emotion estimation module, and interface control module. The emotion estimation module receives user input data (e.g., text “severe pain,” “I don't know what to do,” voice data in WAV format 16 kHz, image data: facial expression images), and uses a natural language processing model (e.g., Transformer-based large language model), voice emotion recognition model (e.g., convolutional neural network+RNN), and image emotion estimation model (e.g., facial expression recognition CNN) to estimate the user's emotional state (e.g., anxiety, urgency, relaxation). Examples of input to the AI include “Text: ‘I'm worried, what should I do?’” “Voice: trembling voice saying ‘Please help’” “Image: facial expression with furrowed brows.” The AI output is structured data such as emotion labels (e.g., anxiety, urgency, relaxation), emotion scores (e.g., anxiety level 0.85, urgency level 0.65), and confidence (e.g., 0.92). The interface control module receives these emotion estimation results and automatically switches the interface color scheme (e.g., calm blue tones), layout (e.g., simplification of input items, emphasis on important items), and guidance display (e.g., reassuring messages such as “Please enter calmly”). In subsequent processing, the emotion estimation results are sent to the user experience optimization engine and input assistance module and used to prevent input errors and enhance input support. As a technical effect, the reception unit eliminates dependence on subjective human observation and empirical rules, realizes high-precision and rapid emotion estimation and interface control by AI, greatly reduces the user's psychological burden, and significantly improves input accuracy, speed, and satisfaction. Specific application fields include emergency response in general households, triage in animal hospitals, remote medical support, response to multiple casualties during disasters, and medical sites requiring psychological care. Unlike conventional human observation and simple UI switching, the present invention uses unconventional and technical methods such as AI inference in high-dimensional feature space, multimodal emotion estimation, and real-time UI optimization, thereby contributing to the improvement of computer technology itself.

The reception unit can analyze the user's past input history and propose an optimal input method. For example, the reception unit automatically displays as candidates information about illnesses or injuries that the user has frequently input in the past. The reception unit can also preferentially propose input methods (voice, text, etc.) that the user has used in the past. Furthermore, the reception unit can predict and propose information to be input at specific times based on the user's past input history. By analyzing the user's past input history, the reception unit can propose an optimal input method and streamline the input operation. Specifically, the reception unit comprises multiple hardware and software elements such as an input history database, history analysis module, input method recommendation module, and interface control module. The history analysis module acquires past input data accumulated for each user (e.g., image data, text data, voice data, input time, input method type) from a time-series database and analyzes the user's input tendencies and preferences using feature extraction algorithms (e.g., time-series clustering, frequency analysis, pattern mining). Examples of input to the AI include “Input history for the past 30 days: 20 image inputs, 15 text inputs, 5 voice inputs,” “Input time: mostly at night on weekdays,” “Frequent symptoms: ‘bleeding from the right foreleg.’” The AI output is structured data such as recommended input methods (e.g., prioritize image input, recommend voice input), candidate input item lists (e.g., “bleeding from the right foreleg,” “swelling”), and input timing predictions (e.g., tendency to input at 7 p.m.). The input method recommendation module receives these outputs and automatically displays recommended input method buttons and candidate item lists on the interface, allowing the user to input with minimal operations. In subsequent processing, the recommendation results are sent to the input assistance engine and history learning module and used for continuous improvement of recommendation accuracy and user experience optimization. As a technical effect, the reception unit eliminates the need for human memory and manual history reference, realizes high-precision and rapid history analysis and input method recommendation by AI, and achieves efficiency in input operations, prevention of erroneous input, and improvement of user satisfaction. Specific application fields include emergency response in general households, triage in animal hospitals, remote medical support, response to multiple casualties during disasters, and regular reporting by chronic disease patients. Unlike conventional human history reference and simple history display, the present invention uses unconventional and technical methods such as AI inference in high-dimensional feature space, time-series pattern analysis, and real-time recommendation, thereby contributing to the improvement of computer technology itself.

The reception unit can customize input items for entering photographs or situations of illness or injury based on the user's current health condition and past medical history. For example, the reception unit refers to the user's past medical history and automatically displays related input items. The reception unit can also prioritize the display of necessary input items based on the user's current health condition. Furthermore, the reception unit can customize input items based on the user's past medical history and current health condition to simplify the input operation. By customizing input items based on the user's current health condition and past medical history, the reception unit can simplify the input operation and obtain accurate information. Specifically, the reception unit comprises multiple hardware and software elements such as a health information database, medical history analysis module, input item generation module, and interface control module. The medical history analysis module acquires structured data such as past medical records, pre-existing conditions, allergy information, and regular medication information accumulated for each user, and estimates current health risks and related symptoms using health condition estimation algorithms (e.g., multilayer perceptron, decision tree, rule-based inference). Examples of input to the AI include “Past medical history: diabetes, hypertension,” “Current health condition: fever, cough,” “Input image: swelling of the leg.” The AI output is structured data such as recommended input item lists (e.g., “blood glucose level,” “medication status,” “location of swelling”), input item priorities (e.g., display blood glucose input at the top), and suggestions for omitting input items (e.g., hide unnecessary items). The input item generation module receives these outputs and automatically generates a customized input form on the interface, allowing the user to efficiently input only the minimum necessary information. In subsequent processing, the customization results are sent to the health management engine and history learning module and used for continuous input optimization and improvement of medical data quality. As a technical effect, the reception unit eliminates the need for human memory and manual medical history reference, realizes high-precision and rapid health condition estimation and input item customization by AI, and achieves simplification of input operations, improvement of information accuracy, and reduction of user burden. Specific application fields include emergency response in general households, triage in animal hospitals, regular reporting by chronic disease patients, remote medical support, and response to multiple casualties during disasters. Unlike conventional human medical history reference and fixed input forms, the present invention uses unconventional and technical methods such as AI inference in high-dimensional feature space, health condition-linked UI generation, and real-time input optimization, thereby contributing to the improvement of computer technology itself.

The reception unit can estimate the user's emotion and determine the priority of input based on the estimated emotion. For example, if the user feels anxious, the reception unit prioritizes the display of important input items and prompts rapid input. If the user is relaxed, the reception unit provides detailed input options and proposes customizable input methods. Furthermore, if the user is in a hurry, the reception unit prioritizes the display of the most important input items and prompts rapid input. By determining the priority of input according to the user's emotion, the reception unit enables rapid input of important information. Emotion estimation is realized by using, for example, an emotion engine or generative AI with emotion estimation functions. Generative AI may be, for example, a text generation AI (such as an LLM) or a multimodal generative AI, but is not limited thereto. Specifically, the reception unit comprises multiple hardware and software elements such as an emotion estimation module, input item priority determination module, and interface control module. The emotion estimation module receives user input data (e.g., text “I'm worried, what should I do?”, voice data in WAV format 16 kHz, facial expression images), and uses a natural language processing model, voice emotion recognition model, and image emotion estimation model to estimate the user's emotional state (e.g., anxiety, urgency, relaxation). Examples of input to the AI include “Text: ‘Please help,’” “Voice: fast and tense speech,” “Image: surprised facial expression.” The AI output is structured data such as emotion labels (e.g., anxiety, urgency, relaxation), emotion scores (e.g., anxiety level 0.92), and confidence. The input item priority determination module receives the emotion estimation results and automatically determines the priority of input items (e.g., display high-urgency items at the top, omit detailed items), and the interface control module switches the UI in real time. In subsequent processing, the priority determination results are sent to the input assistance engine and history learning module and used for continuous optimization and improvement of user experience. As a technical effect, the reception unit eliminates dependence on subjective human observation and empirical rules, realizes high-precision and rapid emotion estimation and input item priority control by AI, and achieves rapid input of important information, prevention of erroneous input, and improvement of user satisfaction. Specific application fields include emergency response in general households, triage in animal hospitals, remote medical support, response to multiple casualties during disasters, and medical sites requiring psychological care. Unlike conventional human observation and fixed input order, the present invention uses unconventional and technical methods such as AI inference in high-dimensional feature space, multimodal emotion estimation, and real-time UI optimization, thereby contributing to the improvement of computer technology itself.

The reception unit can prioritize the input of highly relevant information by considering the user's geographic location when entering photographs or situations of illness or injury. For example, the reception unit prioritizes the display of information about nearby medical institutions based on the user's current location. The reception unit can also prioritize the input of information related to region-specific illnesses or injuries based on the user's geographic location. Furthermore, the reception unit can prioritize the arrangement of the nearest ambulance based on the user's geographic location. By considering the user's geographic location, the reception unit can prioritize the input of highly relevant information and enable rapid response. Specifically, the reception unit comprises multiple hardware and software elements such as a location information acquisition module, geographic information analysis module, input item optimization module, and interface control module. The location information acquisition module uses the GPS function of the user terminal to acquire latitude and longitude information (e.g., latitude 35.6, longitude 139.7). The geographic information analysis module compares the acquired location information with a map database and medical institution information database to extract a list of nearby medical institutions (e.g., hospitals and clinics within a 5 km radius), region-specific disease risks (e.g., heatstroke-prone areas, regions with frequent infectious diseases), and ambulance deployment status (e.g., nearest ambulance ID, estimated arrival time). Examples of input to the AI include “Location information: latitude 35.6, longitude 139.7,” “Input image: insect bite,” “Text: ‘fever.’” The AI output is structured data such as a list of medical institutions to be prioritized for display, input items for region-specific diseases (e.g., heatstroke, infectious diseases), and ambulance arrangement candidates (e.g., ambulance ID123, estimated arrival in 8 minutes). The input item optimization module receives these outputs and automatically displays region-linked input forms and medical institution information on the interface, allowing the user to input information rapidly and appropriately. In subsequent processing, geographic information is sent to the transport destination optimization module and ambulance arrangement engine and used for optimization of the entire medical support process. As a technical effect, the reception unit eliminates the need for human memory and manual geographic information reference, realizes high-precision and rapid geographic information analysis and input item optimization by AI, and achieves rapid response, immediate response to region-specific risks, and improved transport efficiency. Specific application fields include emergency response in general households, triage in animal hospitals, remote medical support, response to multiple casualties during disasters, and regional medical cooperation. Unlike conventional human geographic information reference and fixed input forms, the present invention uses unconventional and technical methods such as AI inference in high-dimensional feature space, geographic information-linked UI generation, and real-time transport optimization, thereby contributing to the improvement of computer technology itself.

The reception unit can analyze the user's social media activity and input relevant information when entering photographs or situations of illness or injury. For example, the reception unit analyzes recent health status and activity from the user's social media posts and proposes relevant input items. The reception unit can also extract information about past illnesses or injuries from the user's social media activity and customize input items. Furthermore, the reception unit can evaluate current health status and disease risk based on the user's social media activity and adjust input items. By analyzing the user's social media activity, the reception unit can input relevant information and obtain accurate information. Specifically, the reception unit comprises multiple hardware and software elements such as a social media integration module, post analysis module, health status estimation module, and input item generation module. The social media integration module acquires post data (e.g., text posts, image posts, post time, location tags) from major SNS or blogs with the user's permission. The post analysis module uses a natural language processing model (e.g., Transformer-based large language model) and image analysis model (e.g., convolutional neural network) to extract descriptions related to health status (e.g., “fever,” “fall,” “traveling”), activity status (e.g., exercise, frequency of going out), and records of past illnesses or injuries from post content. Examples of input to the AI include “SNS post: ‘I've had a fever since yesterday,’ ‘Fell while jogging,’” “Image post: photograph of swollen leg.” The AI output is structured data such as health status estimation (e.g., high fever risk, medium fall risk), recommended input item lists (e.g., “fever,” “location of fall”), and input item customization (e.g., add presence or absence of exercise habits). The input item generation module receives these outputs and automatically displays customized input forms and supplementary questions on the interface, allowing the user to efficiently input accurate information. In subsequent processing, the social media analysis results are sent to the health management engine and history learning module and used to improve the comprehensiveness of medical data and the accuracy of risk assessment. As a technical effect, the reception unit eliminates the need for human memory and manual information collection, realizes high-precision and rapid social media analysis and input item optimization by AI, and achieves improved accuracy and comprehensiveness of information and reduction of user burden. Specific application fields include emergency response in general households, triage in animal hospitals, remote medical support, response to multiple casualties during disasters, and health management applications. Unlike conventional human SNS reference and fixed input forms, the present invention uses unconventional and technical methods such as AI inference in high-dimensional feature space, social media-linked UI generation, and real-time health risk assessment, thereby contributing to the improvement of computer technology itself.

The analysis unit can estimate the user's emotion and adjust the accuracy of analysis based on the estimated emotion. For example, if the user feels anxious, the analysis unit increases the accuracy of analysis and provides detailed analysis results. If the user is relaxed, the analysis unit performs analysis with standard accuracy. Furthermore, if the user is in a hurry, the analysis unit performs rapid analysis and provides results quickly. By adjusting the accuracy of analysis according to the user's emotion, the analysis unit can provide appropriate analysis results. Emotion estimation is realized by using, for example, an emotion engine or generative AI with emotion estimation functions. Generative AI may be, for example, a text generation AI (such as an LLM) or a multimodal generative AI, but is not limited thereto. Specifically, the analysis unit comprises multiple hardware and software elements such as an emotion estimation module, analysis accuracy control module, and multimodal analysis engine. The emotion estimation module receives user input data (e.g., text “I'm worried, what should I do?”, voice data in WAV format 16 kHz, facial expression images), and uses a natural language processing model (e.g., Transformer-based large language model), voice emotion recognition model (e.g., convolutional neural network+RNN), and image emotion estimation model (e.g., facial expression recognition CNN) to estimate the user's emotional state (e.g., anxiety, urgency, relaxation). Examples of input to the AI include “Text: ‘Please help,’” “Voice: trembling voice,” “Image: facial expression with furrowed brows.” The AI output is structured data such as emotion labels (e.g., anxiety, urgency, relaxation), emotion scores (e.g., anxiety level 0.85, urgency level 0.65), and confidence (e.g., 0.92). The analysis accuracy control module receives the emotion estimation results and dynamically adjusts the parameters of the analysis engine (e.g., number of convolutional layers in the image analysis neural network, threshold at inference, detail setting, analysis time constraints). For example, if the user is estimated to be anxious, the image analysis neural network enables multi-stage segmentation and detailed extraction of abnormal sites, and the natural language processing model extracts multiple keywords and time-series progression from symptom descriptions in detail. Conversely, if the user is estimated to be in a hurry, the analysis unit selects a rapid inference mode and extracts only major features to output results quickly. Examples of AI output include “Input: image (bleeding image), text (‘severe pain’), emotion: anxiety→output: severity 0.88, detailed analysis report” and “Input: image (swelling image), text (‘swelling is spreading’), emotion: urgency→output: severity 0.65, simple analysis result.” In subsequent processing, the analysis accuracy control results are sent to the determination unit and notification interface and used for user experience optimization and decision support in medical sites. As a technical effect, the analysis unit eliminates dependence on subjective human observation and empirical rules, realizes high-precision and rapid emotion estimation and analysis accuracy control by AI, provides optimal analysis results according to the user's psychological state, reduces misjudgment and delays, and achieves improved patient safety, satisfaction, and reduced burden on medical sites. Specific application fields include emergency response in general households, triage in animal hospitals, remote medical support, response to multiple casualties during disasters, and medical sites requiring psychological care. Unlike conventional human observation and fixed-accuracy analysis, the present invention uses unconventional and technical methods such as AI inference in high-dimensional feature space, multimodal emotion estimation, and real-time optimization of analysis accuracy, thereby contributing to the improvement of computer technology itself.

The analysis unit can adjust the level of detail of analysis based on the importance of photographs or situations of illness or injury during analysis. For example, in the case of serious illnesses or injuries, the analysis unit performs detailed analysis and provides accurate diagnosis. In the case of minor illnesses or injuries, the analysis unit performs standard analysis and provides rapid results. Furthermore, the analysis unit can adjust the level of detail of analysis according to the importance of the illness or injury and provide appropriate results. By adjusting the level of detail of analysis according to the importance of the illness or injury, the analysis unit can provide appropriate analysis results. Specifically, the analysis unit comprises multiple hardware and software elements such as an importance determination module, analysis detail control module, and multimodal analysis engine. The importance determination module receives image data (e.g., bleeding image, swelling image), text data (e.g., “consciousness disorder,” “massive bleeding”), and voice data (e.g., distressed voice) from the reception unit as input, and uses an image analysis neural network (e.g., convolutional neural network), natural language processing model (e.g., Transformer-based large language model), and voice analysis model to calculate severity scores (e.g., 0.0-1.0) and importance labels (e.g., “serious,” “moderate,” “minor”). Examples of input to the AI include “Image: deep laceration image, text: ‘bleeding does not stop,’” “Image: small abrasion image, text: ‘little pain.’” The AI output is structured data such as severity scores (e.g., 0.92, 0.18) and importance labels (e.g., “serious,” “minor”). The analysis detail control module receives the importance determination results and dynamically adjusts the parameters of the analysis engine (e.g., image analysis resolution, number of feature extraction layers, whether to generate detailed reports, number of inference runs). For example, if judged as serious, the image analysis neural network performs high-resolution input, multi-stage segmentation, and detailed extraction of abnormal sites, and the natural language processing model extracts multiple keywords and time-series progression from symptom descriptions in detail. In the case of minor importance, only major features are extracted and rapid results are output. Examples of AI output include “Serious: detailed analysis report (wound depth map, estimated bleeding volume, complication risk assessment)” and “Minor: simple analysis result (first aid recommended).” In subsequent processing, the analysis detail control results are sent to the determination unit and notification interface and used for decision support in medical sites and user experience optimization. As a technical effect, the analysis unit eliminates dependence on subjective human judgment and empirical rules, realizes high-precision and rapid importance determination and analysis detail control by AI, prevents overlooking of serious cases, reduces misjudgment, reduces burden on medical sites, and streamlines the entire process. Specific application fields include emergency response in general households, triage in animal hospitals, remote medical support, and response to multiple casualties during disasters. Unlike conventional human visual inspection and fixed-detail analysis, the present invention uses unconventional and technical methods such as AI inference in high-dimensional feature space, importance-linked analysis control, and real-time optimization of analysis detail, thereby contributing to the improvement of computer technology itself.

The analysis unit can apply different analysis algorithms according to the category of illness or injury during analysis. For example, in the case of trauma, the analysis unit applies algorithms to analyze wound depth and degree of bleeding. In the case of internal diseases, the analysis unit applies algorithms to analyze details of symptoms. Furthermore, in the case of pet illnesses or injuries, the analysis unit applies algorithms to analyze animal-specific symptoms. By applying different analysis algorithms according to the category of illness or injury, the analysis unit can provide appropriate analysis results. Specifically, the analysis unit comprises multiple hardware and software elements such as a category determination module, algorithm selection module, and multimodal analysis engine. The category determination module receives image data (e.g., trauma images, tumor images, animal images), text data (e.g., “fall,” “fever,” “loss of appetite”), and voice data from the reception unit as input, and uses a natural language processing model and image classification neural network (e.g., ResNet, VGG) to determine category labels (e.g., “trauma,” “internal disease,” “animal disease”). Examples of input to the AI include “Image: bleeding image, text: ‘fell and bleeding,’” “Image: dog swelling image, text: ‘loss of appetite.’” The AI output is structured data such as category labels (e.g., “trauma,” “animal disease”). The algorithm selection module receives the category determination results and automatically switches the analysis engine's algorithm (e.g., trauma image segmentation, internal disease symptom estimation model, animal-specific symptom extraction model). For example, in the case of trauma, the image analysis neural network performs wound depth estimation and bleeding analysis; in the case of internal disease, the natural language processing model extracts detailed symptom progression and complication risk from symptom descriptions; in the case of animal disease, animal species determination models and animal-specific symptom extraction algorithms are applied. Examples of AI output include “Trauma: wound depth map, estimated bleeding volume,” “Internal disease: symptom progression prediction, complication risk assessment,” “Animal disease: species determination, specific symptom extraction.” In subsequent processing, the analysis results are sent to the determination unit and notification interface and used for decision support in medical sites and user experience optimization. As a technical effect, the analysis unit eliminates the need for human experience and manual algorithm selection, realizes high-precision and rapid category determination and automatic algorithm switching by AI, and achieves optimal analysis for each case, reduction of misjudgment, reduction of burden on medical sites, and streamlining of the entire process. Specific application fields include emergency response in general households, triage in animal hospitals, remote medical support, and response to multiple casualties during disasters. Unlike conventional human visual inspection and fixed algorithm application, the present invention uses unconventional and technical methods such as AI inference in high-dimensional feature space, category-linked algorithm selection, and real-time optimization of analysis, thereby contributing to the improvement of computer technology itself.

The analysis unit can estimate the user's emotion and adjust the display method of the analysis result based on the estimated emotion. For example, if the user feels anxious, the analysis unit provides detailed analysis results and reassurance. If the user is relaxed, the analysis unit provides standard analysis results. Furthermore, if the user is in a hurry, the analysis unit provides concise and rapid analysis results. By adjusting the display method of the analysis result according to the user's emotion, the analysis unit can provide results that are easy for the user to understand. Emotion estimation is realized by using, for example, an emotion engine or generative AI with emotion estimation functions. Generative AI may be, for example, a text generation AI (such as an LLM) or a multimodal generative AI, but is not limited thereto. Specifically, the analysis unit comprises multiple hardware and software elements such as an emotion estimation module, display control module, and multimodal analysis engine. The emotion estimation module receives user input data (e.g., text “I'm worried, what should I do?”, voice data in WAV format 16 kHz, facial expression images), and uses a natural language processing model, voice emotion recognition model, and image emotion estimation model to estimate the user's emotional state (e.g., anxiety, urgency, relaxation). Examples of input to the AI include “Text: ‘Please help,’” “Voice: trembling voice,” “Image: anxious facial expression.” The AI output is structured data such as emotion labels (e.g., anxiety, urgency, relaxation), emotion scores (e.g., anxiety level 0.85), and confidence (e.g., 0.92). The display control module receives the emotion estimation results and automatically switches the display method of the analysis result (e.g., detailed report display, simple summary display, reassurance message addition, color scheme and layout changes). For example, if anxiety is estimated, the analysis result is accompanied by detailed explanations and reassurance messages (e.g., “Please remain calm. We will provide a detailed analysis result”), for relaxation, a standard summary display, and for urgency, only the main points are emphasized in a concise display. Examples of AI output include “Detailed analysis report+reassurance message,” “Standard summary,” “Concise main point display.” In subsequent processing, the display control results are sent to the user interface and notification engine and used for user experience optimization and input assistance. As a technical effect, the analysis unit eliminates dependence on subjective human observation and empirical rules, realizes high-precision and rapid emotion estimation and display control by AI, and achieves optimal information presentation, prevention of misunderstanding, and improvement of satisfaction according to the user's psychological state. Specific application fields include emergency response in general households, triage in animal hospitals, remote medical support, response to multiple casualties during disasters, and medical sites requiring psychological care. Unlike conventional human observation and fixed display, the present invention uses unconventional and technical methods such as AI inference in high-dimensional feature space, multimodal emotion estimation, and real-time UI optimization, thereby contributing to the improvement of computer technology itself.

The analysis unit can determine the priority of analysis based on the occurrence time of the illness or injury during analysis. For example, in the case of recently occurred illnesses or injuries, the analysis unit prioritizes analysis and provides rapid results. In the case of illnesses or injuries that occurred in the past, the analysis unit performs standard analysis. Furthermore, the analysis unit can adjust the priority of analysis according to the occurrence time of the illness or injury and provide appropriate results. By determining the priority of analysis according to the occurrence time of the illness or injury, the analysis unit enables rapid response. Specifically, the analysis unit comprises multiple hardware and software elements such as an occurrence time determination module, priority control module, and multimodal analysis engine. The occurrence time determination module analyzes input data received from the reception unit (e.g., image data shooting time, text “fever since this morning,” “fell yesterday,” voice data timestamp) and determines the occurrence time (e.g., within the last hour, within 24 hours, one week ago). Examples of input to the AI include “Image: bleeding image (shooting time: current time−30 minutes), text: ‘pain since this morning,’” “Image: swelling image (shooting time: 2 days ago), text: ‘fell 2 days ago.’” The AI output is structured data such as occurrence time labels (e.g., “recent,” “past”) and priority scores (e.g., 0.95, 0.45). The priority control module receives the occurrence time determination results and automatically adjusts the processing order and resource allocation of the analysis engine (e.g., prioritize immediate analysis for recent cases, batch processing for past cases). For example, in the case of recent occurrence, immediate analysis mode is selected, and for past cases, standard analysis mode is applied. Examples of AI output include “Recent occurrence: immediate analysis, rapid result,” “Past occurrence: standard analysis, detailed report.” In subsequent processing, the priority control results are sent to the determination unit and notification interface and used for decision support in medical sites and user experience optimization. As a technical effect, the analysis unit eliminates the need for human memory and manual time-series management, realizes high-precision and rapid occurrence time determination and priority control by AI, and achieves rapid response to emergency cases, reduction of misjudgment, reduction of burden on medical sites, and streamlining of the entire process. Specific application fields include emergency response in general households, triage in animal hospitals, remote medical support, and response to multiple casualties during disasters. Unlike conventional human time-series management and fixed-order analysis, the present invention uses unconventional and technical methods such as AI inference in high-dimensional feature space, occurrence time-linked priority control, and real-time optimization of analysis, thereby contributing to the improvement of computer technology itself.

The analysis unit can determine the priority of analysis based on the time of occurrence of an illness or injury during analysis. For example, in the case of a recently occurred illness or injury, the analysis unit performs prioritized analysis and provides rapid results. In the case of an illness or injury that occurred in the past, the analysis unit can perform standard analysis. Furthermore, the analysis unit can adjust the priority of analysis according to the time of occurrence of the illness or injury and provide appropriate results. By determining the priority of analysis according to the time of occurrence of the illness or injury, the analysis unit enables prompt response. Specifically, the analysis unit comprises multiple hardware and software elements such as an occurrence time determination module, a priority control module, and a multimodal analysis engine. The occurrence time determination module analyzes input data received from the reception unit (e.g., image data capture time, text such as “fever since this morning,” “fell yesterday,” audio data timestamps) and determines the time of occurrence (e.g., within the last hour, within 24 hours, one week ago, etc.). Examples of AI input include “Image: bleeding image (capture time: current time−30 minutes), text: ‘pain since this morning’” and “Image: swelling image (capture time: 2 days ago), text: ‘fell 2 days ago.’” AI output is structured data such as occurrence time labels (e.g., “recent,” “past”) and priority scores (e.g., 0.95, 0.45). The priority control module receives the occurrence time determination result and automatically adjusts the processing order and resource allocation of the analysis engine (e.g., prioritized analysis for recent cases, batch processing for past cases). For example, immediate analysis mode is selected for recent occurrences, and standard analysis mode is applied for past cases. Examples of AI output include “recent occurrence: immediate analysis, rapid result” and “past occurrence: standard analysis, detailed report.” As subsequent processing, the priority control result is sent to the determination unit and notification interface and used for decision support in medical settings and optimization of user experience. The technical effect is that the analysis unit eliminates human memory and manual chronological management, and achieves high-precision, high-speed occurrence time determination and priority control by AI, thereby enabling rapid response to emergency cases, reducing misjudgments, alleviating the burden on medical sites, and improving overall process efficiency. Specific application fields include emergency response in general households, triage in animal hospitals, remote medical support, and response to multiple casualties in disasters. Unlike conventional human chronological management and fixed-order analysis, the present invention employs unconventional and technical methods such as AI inference in high-dimensional feature space, occurrence time-linked priority control, and real-time analysis optimization, thereby contributing to the improvement of computer technology itself.

The analysis unit can improve the accuracy of analysis by referring to related literature on illnesses or injuries during analysis. For example, the analysis unit refers to the latest research papers on illnesses or injuries to improve the accuracy of analysis. The analysis unit can also refer to past case data to improve analysis accuracy. Furthermore, the analysis unit can optimize analysis algorithms based on related literature on illnesses or injuries to further improve accuracy. Thus, by referring to related literature, the analysis unit can enhance the accuracy of analysis. Specifically, the analysis unit comprises multiple hardware and software elements such as a literature search module, a case database linkage module, and an algorithm optimization module. The literature search module searches and retrieves the latest research papers and case reports related to the input symptoms or images from online medical literature databases and in-hospital case databases. Examples of AI input include “Text: ‘novel virus infection,’ ‘fever and cough’” and “Image: skin rash image.” AI output is structured data such as a list of related literature (e.g., paper title, abstract, publication year) and case data (e.g., past diagnosis results, treatment course, prognosis information). The algorithm optimization module reflects the obtained literature and case data in the parameters of the analysis algorithm (e.g., thresholds, feature selection, weighting, etc.), and performs model retraining and dynamic optimization of inference logic. For example, when the latest treatment methods or diagnostic criteria are presented in papers, the analysis unit automatically updates the algorithm to improve analysis accuracy. Examples of AI output include “Related paper: ‘Skin symptoms of novel virus infection’” and “Case data: diagnosis results of 100 past cases.” As subsequent processing, the results of literature and case data reference are sent to the determination unit and medical staff notification engine and used for improving diagnostic accuracy and supporting treatment policy decisions. The technical effect is that the analysis unit eliminates human memory and manual literature reference, and achieves high-precision, high-speed literature search and algorithm optimization by AI, thereby enabling rapid reflection of the latest knowledge, reducing misjudgments, alleviating the burden on medical sites, and improving overall process efficiency. Specific application fields include emergency response in general households, triage in animal hospitals, remote medical support, response to multiple casualties in disasters, and support for diagnosis of rare diseases. Unlike conventional human literature reference and fixed algorithm application, the present invention employs unconventional and technical methods such as AI inference in high-dimensional feature space, literature-linked algorithm optimization, and real-time knowledge updating, thereby contributing to the improvement of computer technology itself.

The determination unit can estimate the user's emotion and adjust the criteria for determining the necessity of medical consultation based on the estimated emotion. For example, if the user is feeling anxious, the determination unit applies cautious criteria and recommends medical consultation. If the user is relaxed, the determination unit applies standard criteria. Furthermore, if the user is feeling rushed, the determination unit applies rapid criteria and recommends prompt medical consultation. Thus, by adjusting the criteria for determining the necessity of medical consultation according to the user's emotion, the determination unit can make appropriate determinations. Emotion estimation is realized, for example, by using an emotion engine or generative AI with emotion estimation functions. Generative AI may include text generation AI (e.g., LLM) or multimodal generative AI, but is not limited to these examples. Specifically, the determination unit comprises multiple hardware and software elements such as an emotion estimation module, a criteria control module, a severity determination engine, and an interface control module. The emotion estimation module receives user input data (e.g., text such as “What should I do, I'm worried,” audio data in WAV format 16 kHz, facial expression images, etc.) and uses natural language processing models (e.g., Transformer-based large language models), audio emotion recognition models (e.g., convolutional neural network+RNN), and image emotion estimation models (e.g., facial expression recognition CNN) to estimate the user's emotional state (e.g., anxiety, urgency, relaxation, etc.). Examples of AI input include “Text: ‘Please help me,’” “Audio: trembling voice,” and “Image: anxious facial expression.” AI output is structured data such as emotion labels (e.g., anxiety, urgency, relaxation), emotion scores (e.g., anxiety level 0.85, urgency level 0.65), and confidence (e.g., 0.92). The criteria control module receives the emotion estimation result and dynamically adjusts the parameters of the severity determination engine (e.g., consultation recommendation threshold, urgency determination logic, explanation generation pattern, etc.). For example, if anxiety is estimated, the severity score threshold is set lower and a cautious consultation recommendation logic is applied. For relaxation, standard thresholds and logic are applied, and for urgency, rapid determination mode is selected and prompt consultation recommendation is made using only major features. Examples of AI output include “Input: severity 0.62, emotion: anxiety→Output: consultation recommended,” “Input: severity 0.62, emotion: relaxation→Output: home first aid recommended,” and “Input: severity 0.62, emotion: urgency→Output: prompt consultation recommended.” As subsequent processing, the criteria control result is sent to the request unit and notification interface and used for optimizing user experience and supporting decision-making in medical settings. The technical effect is that the determination unit does not depend on human subjective observation or empirical rules, and achieves high-precision, high-speed emotion estimation and criteria control by AI, thereby enabling optimal consultation determination according to the user's psychological state, reducing misjudgments, and improving satisfaction. Specific application fields include emergency response in general households, triage in animal hospitals, remote medical support, response to multiple casualties in disasters, and medical settings requiring psychological care. Unlike conventional human observation and fixed criteria determination, the present invention employs unconventional and technical methods such as AI inference in high-dimensional feature space, multimodal emotion estimation, and real-time criteria optimization, thereby contributing to the improvement of computer technology itself.

The determination unit can adjust the level of detail of determination based on the importance of the condition of the illness or injury during determination. For example, in the case of a serious illness or injury, the determination unit performs detailed determination and provides accurate necessity of medical consultation. In the case of a minor illness or injury, the determination unit performs standard determination and provides rapid results. Furthermore, the determination unit can adjust the level of detail of determination according to the importance of the illness or injury and provide appropriate results. Thus, by adjusting the level of detail of determination according to the importance of the illness or injury, the determination unit can make appropriate determinations. Specifically, the determination unit comprises multiple hardware and software elements such as an importance determination module, a detail control module, a severity determination engine, and an interface control module. The importance determination module receives image data (e.g., bleeding images, swelling images), text data (e.g., “consciousness disorder,” “massive bleeding,” etc.), and audio data (e.g., distressed voice) from the analysis unit, and uses image analysis neural networks, natural language processing models, and audio analysis models to calculate severity scores (e.g., 0.0 to 1.0) and importance labels (e.g., “serious,” “moderate,” “minor”). Examples of AI input include “Image: deep laceration image, text: ‘bleeding does not stop’” and “Image: small abrasion image, text: ‘little pain.’” AI output is structured data such as severity scores (e.g., 0.92, 0.18) and importance labels (e.g., “serious,” “minor”). The detail control module receives the importance determination result and dynamically adjusts the parameters of the severity determination engine (e.g., number of logic branches, level of detail in explanation generation, presence of additional questions, etc.). For example, if determined as serious, multiple criteria, detailed explanations, and additional questions are enabled; if minor, rapid determination is performed using only major features. Examples of AI output include “Serious: detailed determination+additional questions presented” and “Minor: simple determination, first aid recommended.” As subsequent processing, the detail control result is sent to the request unit and notification interface and used for decision support in medical settings and optimization of user experience. The technical effect is that the determination unit does not depend on human subjective judgment or empirical rules, and achieves high-precision, high-speed importance determination and detail control by AI, thereby preventing oversight of serious cases, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed detail determination, the present invention employs unconventional and technical methods such as AI inference in high-dimensional feature space, importance-linked determination control, and real-time detail optimization, thereby contributing to the improvement of computer technology itself.

The determination unit can apply different determination algorithms according to the category of illness or injury during determination. For example, in the case of trauma, the determination unit applies an algorithm that determines based on the depth of the wound and the degree of bleeding. In the case of internal diseases, the determination unit applies an algorithm that determines based on the details of symptoms. Furthermore, in the case of pet illnesses or injuries, the determination unit applies an algorithm that determines based on animal-specific symptoms. Thus, by applying different determination algorithms according to the category of illness or injury, the determination unit can make appropriate determinations. Specifically, the determination unit comprises multiple hardware and software elements such as a category determination module, an algorithm selection module, and a severity determination engine. The category determination module receives image data (e.g., trauma images, tumor images, animal images), text data (e.g., “fall,” “fever,” “loss of appetite,” etc.), and audio data from the analysis unit, and uses natural language processing models and image classification neural networks (e.g., ResNet, VGG, etc.) to determine category labels (e.g., “trauma,” “internal disease,” “animal disease,” etc.). Examples of AI input include “Image: bleeding image, text: ‘fell and bleeding’” and “Image: dog swelling image, text: ‘loss of appetite.’” AI output is structured data such as category labels (e.g., “trauma,” “animal disease”). The algorithm selection module receives the category determination result and automatically switches the algorithm of the severity determination engine (e.g., trauma determination logic, internal disease determination logic, animal-specific symptom determination logic, etc.). For example, in the case of trauma, wound depth estimation and bleeding volume analysis are emphasized; in the case of internal disease, symptom progression and complication risk are emphasized; in the case of animal disease, species determination and extraction of specific symptoms are performed. Examples of AI output include “Trauma: consultation recommended+wound depth explanation,” “Internal disease: observation recommended+complication risk presented,” and “Animal disease: animal hospital consultation recommended+species explanation.” As subsequent processing, the determination result is sent to the request unit and notification interface and used for decision support in medical settings and optimization of user experience. The technical effect is that the determination unit eliminates human empirical rules and manual algorithm selection, and achieves high-precision, high-speed category determination and automatic algorithm switching by AI, thereby enabling optimal determination for each case, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed algorithm application, the present invention employs unconventional and technical methods such as AI inference in high-dimensional feature space, category-linked algorithm selection, and real-time determination optimization, thereby contributing to the improvement of computer technology itself.

The determination unit can estimate the user's emotion and adjust the display method of the determination result based on the estimated emotion. For example, if the user is feeling anxious, the determination unit provides detailed determination results to give reassurance. If the user is relaxed, the determination unit provides standard determination results. Furthermore, if the user is feeling rushed, the determination unit provides concise and rapid determination results. Thus, by adjusting the display method of the determination result according to the user's emotion, the determination unit can provide results that are easy for the user to understand. Emotion estimation is realized, for example, by using an emotion engine or generative AI with emotion estimation functions. Generative AI may include text generation AI (e.g., LLM) or multimodal generative AI, but is not limited to these examples. Specifically, the determination unit comprises multiple hardware and software elements such as an emotion estimation module, a display control module, and a severity determination engine. The emotion estimation module receives user input data (e.g., text such as “What should I do, I'm worried,” audio data in WAV format 16 kHz, facial expression images, etc.) and uses natural language processing models, audio emotion recognition models, and image emotion estimation models to estimate the user's emotional state (e.g., anxiety, urgency, relaxation, etc.). Examples of AI input include “Text: ‘Please help me,’” “Audio: trembling voice,” and “Image: anxious facial expression.” AI output is structured data such as emotion labels (e.g., anxiety, urgency, relaxation), emotion scores (e.g., anxiety level 0.85, etc.), and confidence (e.g., 0.92). The display control module receives the emotion estimation result and automatically switches the display method of the determination result (e.g., detailed report display, simple summary display, reassurance message addition, color tone/layout change, etc.). For example, if anxiety is estimated, detailed explanations and reassurance messages (e.g., “Please stay calm. We will provide detailed determination results.”) are added to the determination result; for relaxation, standard summary display is applied; for urgency, only key points are emphasized in a concise manner. Examples of AI output include “Detailed determination report+reassurance message,” “Standard summary,” and “Concise key point display.” As subsequent processing, the display control result is sent to the user interface and notification engine and used for optimizing user experience and input assistance. The technical effect is that the determination unit does not depend on human subjective observation or empirical rules, and achieves high-precision, high-speed emotion estimation and display control by AI, thereby enabling optimal information presentation according to the user's psychological state, preventing misunderstandings, and improving satisfaction. Specific application fields include emergency response in general households, triage in animal hospitals, remote medical support, response to multiple casualties in disasters, and medical settings requiring psychological care. Unlike conventional human observation and fixed display, the present invention employs unconventional and technical methods such as AI inference in high-dimensional feature space, multimodal emotion estimation, and real-time UI optimization, thereby contributing to the improvement of computer technology itself.

The determination unit can determine the priority of determination based on the time of occurrence of an illness or injury during determination. For example, in the case of a recently occurred illness or injury, the determination unit performs prioritized determination and provides rapid results. In the case of an illness or injury that occurred in the past, the determination unit can perform standard determination. Furthermore, the determination unit can adjust the priority of determination according to the time of occurrence of the illness or injury and provide appropriate results. By determining the priority of determination according to the time of occurrence of the illness or injury, the determination unit enables prompt response. Specifically, the determination unit comprises multiple hardware and software elements such as an occurrence time determination module, a priority control module, and a severity determination engine. The occurrence time determination module analyzes input data received from the analysis unit (e.g., image data capture time, text such as “fever since this morning,” “fell yesterday,” audio data timestamps) and determines the time of occurrence (e.g., within the last hour, within 24 hours, one week ago, etc.). Examples of AI input include “Image: bleeding image (capture time: current time−30 minutes), text: ‘pain since this morning’” and “Image: swelling image (capture time: 2 days ago), text: ‘fell 2 days ago.’” AI output is structured data such as occurrence time labels (e.g., “recent,” “past”) and priority scores (e.g., 0.95, 0.45). The priority control module receives the occurrence time determination result and automatically adjusts the processing order and resource allocation of the severity determination engine (e.g., prioritized determination for recent cases, batch processing for past cases). For example, immediate determination mode is selected for recent occurrences, and standard determination mode is applied for past cases. Examples of AI output include “recent occurrence: immediate determination, rapid result” and “past occurrence: standard determination, detailed report.” As subsequent processing, the priority control result is sent to the request unit and notification interface and used for decision support in medical settings and optimization of user experience. The technical effect is that the determination unit eliminates human memory and manual chronological management, and achieves high-precision, high-speed occurrence time determination and priority control by AI, thereby enabling rapid response to emergency cases, reducing misjudgments, alleviating the burden on medical sites, and improving overall process efficiency. Specific application fields include emergency response in general households, triage in animal hospitals, remote medical support, and response to multiple casualties in disasters. Unlike conventional human chronological management and fixed-order determination, the present invention employs unconventional and technical methods such as AI inference in high-dimensional feature space, occurrence time-linked priority control, and real-time determination optimization, thereby contributing to the improvement of computer technology itself.

The determination unit can improve the accuracy of determination by referring to related literature on illnesses or injuries during determination. For example, the determination unit refers to the latest research papers on illnesses or injuries to improve the accuracy of determination. The determination unit can also refer to past case data to improve determination accuracy. Furthermore, the determination unit can optimize determination algorithms based on related literature on illnesses or injuries to further improve accuracy. Thus, by referring to related literature, the determination unit can enhance the accuracy of determination. Specifically, the determination unit comprises multiple hardware and software elements such as a literature search module, a case database linkage module, and an algorithm optimization module. The literature search module searches and retrieves the latest research papers and case reports related to symptoms or images extracted by the analysis unit from online medical literature databases and in-hospital case databases. Examples of AI input include “Text: ‘novel virus infection,’ ‘fever and cough’” and “Image: skin rash image.” AI output is structured data such as a list of related literature (e.g., paper title, abstract, publication year) and case data (e.g., past diagnosis results, treatment course, prognosis information). The algorithm optimization module reflects the obtained literature and case data in the parameters of the determination algorithm (e.g., thresholds, feature selection, weighting, etc.), and performs model retraining and dynamic optimization of inference logic. For example, when the latest treatment methods or diagnostic criteria are presented in papers, the determination unit automatically updates the algorithm to improve determination accuracy. Examples of AI output include “Related paper: ‘Skin symptoms of novel virus infection’” and “Case data: diagnosis results of 100 past cases.” As subsequent processing, the results of literature and case data reference are sent to the request unit and medical staff notification engine and used for improving diagnostic accuracy and supporting treatment policy decisions. The technical effect is that the determination unit eliminates human memory and manual literature reference, and achieves high-precision, high-speed literature search and algorithm optimization by AI, thereby enabling rapid reflection of the latest knowledge, reducing misjudgments, alleviating the burden on medical sites, and improving overall process efficiency. Specific application fields include emergency response in general households, triage in animal hospitals, remote medical support, response to multiple casualties in disasters, and support for diagnosis of rare diseases. Unlike conventional human literature reference and fixed algorithm application, the present invention employs unconventional and technical methods such as AI inference in high-dimensional feature space, literature-linked algorithm optimization, and real-time knowledge updating, thereby contributing to the improvement of computer technology itself.

The request unit can estimate the user's emotion and adjust the method of requesting an ambulance based on the estimated emotion. For example, if the user is feeling anxious, the request unit provides detailed information to give reassurance. If the user is relaxed, the request unit provides a standard request method. Furthermore, if the user is feeling rushed, the request unit provides a rapid request method and prompts prompt response. Thus, by adjusting the method of requesting an ambulance according to the user's emotion, the request unit enables rapid and appropriate response. Emotion estimation is realized, for example, by using an emotion engine or generative AI with emotion estimation functions. Generative AI may include text generation AI (e.g., LLM) or multimodal generative AI, but is not limited to these examples. Specifically, the request unit comprises multiple hardware and software elements such as an emotion estimation module, a request method control module, an ambulance arrangement engine, and an interface control module. The emotion estimation module receives user input data (e.g., text such as “What should I do, I'm worried,” audio data in WAV format 16 kHz, facial expression images, etc.) and uses natural language processing models (e.g., Transformer-based large language models), audio emotion recognition models (e.g., convolutional neural network+RNN), and image emotion estimation models (e.g., facial expression recognition CNN) to estimate the user's emotional state (e.g., anxiety, urgency, relaxation, etc.). Examples of AI input include “Text: ‘Please help me,’” “Audio: trembling voice,” and “Image: anxious facial expression.” AI output is structured data such as emotion labels (e.g., anxiety, urgency, relaxation), emotion scores (e.g., anxiety level 0.85, urgency level 0.65), and confidence (e.g., 0.92). The request method control module receives the emotion estimation result and dynamically switches the request method of the ambulance arrangement engine (e.g., detailed information addition, standard request, simple request, guidance display, request flow branching, etc.). For example, if anxiety is estimated, the request unit automatically adds “detailed explanation of current situation,” “display of reassurance message,” and “sequential notification of progress” when requesting an ambulance. For relaxation, the standard request flow is applied; for urgency, immediate request is executed with minimal input, shortening the time to request completion. Examples of AI output include “Detailed request: patient condition, location information, reassurance message added,” “Standard request: only necessary information sent,” and “Simple request: immediate arrangement, progress notification omitted.” As subsequent processing, the request method control result is sent to the interface control module and notification engine and used for optimizing user experience and input assistance. The technical effect is that the request unit does not depend on human subjective observation or empirical rules, and achieves high-precision, high-speed emotion estimation and request method control by AI, thereby enabling optimal ambulance request according to the user's psychological state, preventing misunderstandings, and improving satisfaction. Specific application fields include emergency response in general households, triage in animal hospitals, remote medical support, response to multiple casualties in disasters, and medical settings requiring psychological care. Unlike conventional human observation and fixed request flow, the present invention employs unconventional and technical methods such as AI inference in high-dimensional feature space, multimodal emotion estimation, and real-time request method optimization, thereby contributing to the improvement of computer technology itself.

The request unit can adjust the level of detail of request based on the importance of the condition of the illness or injury during request. For example, in the case of a serious illness or injury, the request unit provides detailed information and prompts rapid response. In the case of a minor illness or injury, the request unit provides a standard request method. Furthermore, the request unit can adjust the level of detail of request according to the importance of the illness or injury and prompt appropriate response. Thus, by adjusting the level of detail of request according to the importance of the illness or injury, the request unit enables rapid and appropriate response. Specifically, the request unit comprises multiple hardware and software elements such as an importance determination module, a request detail control module, and an ambulance arrangement engine. The importance determination module receives image data (e.g., bleeding images, swelling images), text data (e.g., “consciousness disorder,” “massive bleeding,” etc.), and audio data (e.g., distressed voice) from the analysis unit, and uses image analysis neural networks, natural language processing models, and audio analysis models to calculate severity scores (e.g., 0.0 to 1.0) and importance labels (e.g., “serious,” “moderate,” “minor”). Examples of AI input include “Image: deep laceration image, text: ‘bleeding does not stop’” and “Image: small abrasion image, text: ‘little pain.’” AI output is structured data such as severity scores (e.g., 0.92, 0.18) and importance labels (e.g., “serious,” “minor”). The request detail control module receives the importance determination result and dynamically adjusts the request detail of the ambulance arrangement engine (e.g., detailed information addition, presence of additional questions, frequency of progress notifications, etc.). For example, if determined as serious, detailed information such as patient condition, vital signs, candidate hospitals for transport, and necessary medical resources are automatically added; if minor, rapid request is made with only major information. Examples of AI output include “Serious: detailed request+additional information presented” and “Minor: simple request, first aid recommended.” As subsequent processing, the request detail control result is sent to the interface control module and notification engine and used for decision support in medical settings and optimization of user experience. The technical effect is that the request unit does not depend on human subjective judgment or empirical rules, and achieves high-precision, high-speed importance determination and request detail control by AI, thereby preventing oversight of serious cases, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed detail request, the present invention employs unconventional and technical methods such as AI inference in high-dimensional feature space, importance-linked request control, and real-time detail optimization, thereby contributing to the improvement of computer technology itself.

The request unit can apply different request algorithms according to the category of illness or injury during request. For example, in the case of trauma, the request unit applies an algorithm that requests based on the degree of bleeding and wound depth. In the case of internal diseases, the request unit applies an algorithm that requests based on the details of symptoms. Furthermore, in the case of pet illnesses or injuries, the request unit applies an algorithm that requests based on animal-specific symptoms. Thus, by applying different request algorithms according to the category of illness or injury, the request unit enables rapid and appropriate response. Specifically, the request unit comprises multiple hardware and software elements such as a category determination module, an algorithm selection module, and an ambulance arrangement engine. The category determination module receives image data (e.g., trauma images, tumor images, animal images), text data (e.g., “fall,” “fever,” “loss of appetite,” etc.), and audio data from the analysis unit, and uses natural language processing models and image classification neural networks (e.g., ResNet, VGG, etc.) to determine category labels (e.g., “trauma,” “internal disease,” “animal disease,” etc.). Examples of AI input include “Image: bleeding image, text: ‘fell and bleeding’” and “Image: dog swelling image, text: ‘loss of appetite.’” AI output is structured data such as category labels (e.g., “trauma,” “animal disease”). The algorithm selection module receives the category determination result and automatically switches the request algorithm of the ambulance arrangement engine (e.g., trauma request logic, internal disease request logic, animal-specific symptom request logic, etc.). For example, in the case of trauma, wound depth estimation and bleeding volume analysis are emphasized; in the case of internal disease, symptom progression and complication risk are emphasized; in the case of animal disease, species determination and extraction of specific symptoms are performed. Examples of AI output include “Trauma: detailed request+wound depth explanation,” “Internal disease: observation request+complication risk presented,” and “Animal disease: animal hospital request+species explanation.” As subsequent processing, the request result is sent to the interface control module and notification engine and used for decision support in medical settings and optimization of user experience. The technical effect is that the request unit eliminates human empirical rules and manual algorithm selection, and achieves high-precision, high-speed category determination and automatic algorithm switching by AI, thereby enabling optimal request for each case, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed algorithm application, the present invention employs unconventional and technical methods such as AI inference in high-dimensional feature space, category-linked algorithm selection, and real-time request optimization, thereby contributing to the improvement of computer technology itself.

The request unit can estimate the user's emotion and adjust the display method of the request result based on the estimated emotion. For example, if the user is feeling anxious, the request unit provides detailed request results to give reassurance. If the user is relaxed, the request unit provides standard request results. Furthermore, if the user is feeling rushed, the request unit provides concise and rapid request results. Thus, by adjusting the display method of the request result according to the user's emotion, the request unit can provide results that are easy for the user to understand. Emotion estimation is realized, for example, by using an emotion engine or generative AI with emotion estimation functions. Generative AI may include text generation AI (e.g., LLM) or multimodal generative AI, but is not limited to these examples. Specifically, the request unit comprises multiple hardware and software elements such as an emotion estimation module, a display control module, and an ambulance arrangement engine. The emotion estimation module receives user input data (e.g., text such as “What should I do, I'm worried,” audio data in WAV format 16 kHz, facial expression images, etc.) and uses natural language processing models, audio emotion recognition models, and image emotion estimation models to estimate the user's emotional state (e.g., anxiety, urgency, relaxation, etc.). Examples of AI input include “Text: ‘Please help me,’” “Audio: trembling voice,” and “Image: anxious facial expression.” AI output is structured data such as emotion labels (e.g., anxiety, urgency, relaxation), emotion scores (e.g., anxiety level 0.85, etc.), and confidence (e.g., 0.92). The display control module receives the emotion estimation result and automatically switches the display method of the request result (e.g., detailed report display, simple summary display, reassurance message addition, color tone/layout change, etc.). For example, if anxiety is estimated, detailed explanations and reassurance messages (e.g., “Please stay calm. The ambulance arrangement has been completed.”) are added to the request result; for relaxation, standard summary display is applied; for urgency, only key points are emphasized in a concise manner. Examples of AI output include “Detailed request report+reassurance message,” “Standard summary,” and “Concise key point display.” As subsequent processing, the display control result is sent to the user interface and notification engine and used for optimizing user experience and input assistance. The technical effect is that the request unit does not depend on human subjective observation or empirical rules, and achieves high-precision, high-speed emotion estimation and display control by AI, thereby enabling optimal information presentation according to the user's psychological state, preventing misunderstandings, and improving satisfaction. Specific application fields include emergency response in general households, triage in animal hospitals, remote medical support, response to multiple casualties in disasters, and medical settings requiring psychological care. Unlike conventional human observation and fixed display, the present invention employs unconventional and technical methods such as AI inference in high-dimensional feature space, multimodal emotion estimation, and real-time UI optimization, thereby contributing to the improvement of computer technology itself.

The request unit can determine the priority of request based on the time of occurrence of an illness or injury during request. For example, in the case of a recently occurred illness or injury, the request unit performs prioritized request and prompts rapid response. In the case of an illness or injury that occurred in the past, the request unit can perform standard request. Furthermore, the request unit can adjust the priority of request according to the time of occurrence of the illness or injury and prompt appropriate response. By determining the priority of request according to the time of occurrence of the illness or injury, the request unit enables prompt response. Specifically, the request unit comprises multiple hardware and software elements such as an occurrence time determination module, a priority control module, and an ambulance arrangement engine. The occurrence time determination module analyzes input data received from the analysis unit (e.g., image data capture time, text such as “fever since this morning,” “fell yesterday,” audio data timestamps) and determines the time of occurrence (e.g., within the last hour, within 24 hours, one week ago, etc.). Examples of AI input include “Image: bleeding image (capture time: current time−30 minutes), text: ‘pain since this morning’” and “Image: swelling image (capture time: 2 days ago), text: ‘fell 2 days ago.’” AI output is structured data such as occurrence time labels (e.g., “recent,” “past”) and priority scores (e.g., 0.95, 0.45). The priority control module receives the occurrence time determination result and automatically adjusts the request order and resource allocation of the ambulance arrangement engine (e.g., prioritized request for recent cases, batch processing for past cases). For example, immediate request mode is selected for recent occurrences, and standard request mode is applied for past cases. Examples of AI output include “recent occurrence: immediate request, rapid result” and “past occurrence: standard request, detailed report.” As subsequent processing, the priority control result is sent to the interface control module and notification engine and used for decision support in medical settings and optimization of user experience. The technical effect is that the request unit eliminates human memory and manual chronological management, and achieves high-precision, high-speed occurrence time determination and priority control by AI, thereby enabling rapid response to emergency cases, reducing misjudgments, alleviating the burden on medical sites, and improving overall process efficiency. Specific application fields include emergency response in general households, triage in animal hospitals, remote medical support, and response to multiple casualties in disasters. Unlike conventional human chronological management and fixed-order request, the present invention employs unconventional and technical methods such as AI inference in high-dimensional feature space, occurrence time-linked priority control, and real-time request optimization, thereby contributing to the improvement of computer technology itself.

The request unit can improve the accuracy of request by referring to related literature on illnesses or injuries during request. For example, the request unit refers to the latest research papers on illnesses or injuries to improve the accuracy of request. The request unit can also refer to past case data to improve request accuracy. Furthermore, the request unit can optimize request algorithms based on related literature on illnesses or injuries to further improve accuracy. Thus, by referring to related literature, the request unit can enhance the accuracy of request. Specifically, the request unit comprises multiple hardware and software elements such as a literature search module, a case database linkage module, and an algorithm optimization module. The literature search module searches and retrieves the latest research papers and case reports related to symptoms or images extracted by the analysis unit from online medical literature databases and in-hospital case databases. Examples of AI input include “Text: ‘novel virus infection,’ ‘fever and cough’” and “Image: skin rash image.” AI output is structured data such as a list of related literature (e.g., paper title, abstract, publication year) and case data (e.g., past diagnosis results, treatment course, prognosis information). The algorithm optimization module reflects the obtained literature and case data in the parameters of the request algorithm (e.g., thresholds, feature selection, weighting, etc.), and performs model retraining and dynamic optimization of inference logic. For example, when the latest treatment methods or diagnostic criteria are presented in papers, the request unit automatically updates the algorithm to improve request accuracy. Examples of AI output include “Related paper: ‘Skin symptoms of novel virus infection’” and “Case data: diagnosis results of 100 past cases.” As subsequent processing, the results of literature and case data reference are sent to the interface control module and medical staff notification engine and used for improving diagnostic accuracy and supporting treatment policy decisions. The technical effect is that the request unit eliminates human memory and manual literature reference, and achieves high-precision, high-speed literature search and algorithm optimization by AI, thereby enabling rapid reflection of the latest knowledge, reducing misjudgments, alleviating the burden on medical sites, and improving overall process efficiency. Specific application fields include emergency response in general households, triage in animal hospitals, remote medical support, response to multiple casualties in disasters, and support for diagnosis of rare diseases. Unlike conventional human literature reference and fixed algorithm application, the present invention employs unconventional and technical methods such as AI inference in high-dimensional feature space, literature-linked algorithm optimization, and real-time knowledge updating, thereby contributing to the improvement of computer technology itself.

The provisional request unit can estimate the user's emotion and adjust the method of provisional request to a hospital based on the estimated emotion. For example, if the user is feeling anxious, the provisional request unit provides detailed information to give reassurance. If the user is relaxed, the provisional request unit provides a standard provisional request method. Furthermore, if the user is feeling rushed, the provisional request unit provides a rapid provisional request method and prompts prompt response. Thus, by adjusting the method of provisional request to a hospital according to the user's emotion, the provisional request unit enables rapid and appropriate response. Emotion estimation is realized, for example, by using an emotion engine or generative AI with emotion estimation functions. Generative AI may include text generation AI (e.g., LLM) or multimodal generative AI, but is not limited to these examples. Specifically, the provisional request unit comprises multiple hardware and software elements such as an emotion estimation module, a provisional request method control module, a hospital arrangement engine, and an interface control module. The emotion estimation module receives user input data (e.g., text such as “I am very anxious,” audio data in WAV format 16 kHz, facial expression images, etc.) and uses natural language processing models (e.g., Transformer-based large language models), audio emotion recognition models (e.g., convolutional neural network+RNN), and image emotion estimation models (e.g., facial expression recognition CNN) to estimate the user's emotional state (e.g., anxiety, urgency, relaxation, etc.). Examples of AI input include “Text: ‘What should I do, I'm worried,’” “Audio: ‘Please help me’ in a trembling voice,” and “Image: facial expression with furrowed brow.” AI output is structured data such as emotion labels (e.g., anxiety, urgency, relaxation), emotion scores (e.g., anxiety level 0.85, urgency level 0.65), and confidence (e.g., 0.92). The provisional request method control module receives the emotion estimation result and dynamically switches the provisional request method of the hospital arrangement engine (e.g., detailed information addition, standard provisional request, simple provisional request, guidance display, provisional request flow branching, etc.). For example, if anxiety is estimated, the provisional request unit automatically adds “detailed explanation of current situation,” “display of reassurance message,” and “sequential notification of progress” when making a provisional request to a hospital. For relaxation, the standard provisional request flow is applied; for urgency, immediate provisional request is executed with minimal input, shortening the time to completion. Examples of AI output include “Detailed provisional request: patient condition, location information, reassurance message added,” “Standard provisional request: only necessary information sent,” and “Simple provisional request: immediate arrangement, progress notification omitted.” As subsequent processing, the provisional request method control result is sent to the interface control module and notification engine and used for optimizing user experience and input assistance. The technical effect is that the provisional request unit does not depend on human subjective observation or empirical rules, and achieves high-precision, high-speed emotion estimation and provisional request method control by AI, thereby enabling optimal provisional hospital request according to the user's psychological state, preventing misunderstandings, and improving satisfaction. Specific application fields include emergency response in general households, triage in animal hospitals, remote medical support, response to multiple casualties in disasters, and medical settings requiring psychological care. Unlike conventional human observation and fixed provisional request flow, the present invention employs unconventional and technical methods such as AI inference in high-dimensional feature space, multimodal emotion estimation, and real-time provisional request method optimization, thereby contributing to the improvement of computer technology itself.

The provisional request unit can adjust the level of detail of provisional request based on the importance of the condition of the illness or injury during provisional request. For example, in the case of a serious illness or injury, the provisional request unit provides detailed information and prompts rapid response. In the case of a minor illness or injury, the provisional request unit provides a standard provisional request method. Furthermore, the provisional request unit can adjust the level of detail of provisional request according to the importance of the illness or injury and prompt appropriate response. Thus, by adjusting the level of detail of provisional request according to the importance of the illness or injury, the provisional request unit enables rapid and appropriate response. Specifically, the provisional request unit comprises multiple hardware and software elements such as an importance determination module, a provisional request detail control module, and a hospital arrangement engine. The importance determination module receives image data (e.g., bleeding images, swelling images), text data (e.g., “consciousness disorder,” “massive bleeding,” etc.), and audio data (e.g., distressed voice) from the reception unit or analysis unit, and uses image analysis neural networks (e.g., convolutional neural networks), natural language processing models (e.g., Transformer-based large language models), and audio analysis models to calculate severity scores (e.g., 0.0 to 1.0) and importance labels (e.g., “serious,” “moderate,” “minor”). Examples of AI input include “Image: deep laceration image, text: ‘bleeding does not stop’” and “Image: small abrasion image, text: ‘little pain.’” AI output is structured data such as severity scores (e.g., 0.92, 0.18) and importance labels (e.g., “serious,” “minor”). The provisional request detail control module receives the importance determination result and dynamically adjusts the provisional request detail of the hospital arrangement engine (e.g., detailed information addition, presence of additional questions, frequency of progress notifications, etc.). For example, if determined as serious, detailed information such as patient condition, vital signs, candidate hospitals for transport, and necessary medical resources are automatically added; if minor, rapid provisional request is made with only major information. Examples of AI output include “Serious: detailed provisional request+additional information presented” and “Minor: simple provisional request, first aid recommended.” As subsequent processing, the provisional request detail control result is sent to the interface control module and notification engine and used for decision support in medical settings and optimization of user experience. The technical effect is that the provisional request unit does not depend on human subjective judgment or empirical rules, and achieves high-precision, high-speed importance determination and provisional request detail control by AI, thereby preventing oversight of serious cases, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed detail provisional request, the present invention employs unconventional and technical methods such as AI inference in high-dimensional feature space, importance-linked provisional request control, and real-time detail optimization, thereby contributing to the improvement of computer technology itself.

The provisional request unit can apply different provisional request algorithms according to the category of illness or injury during provisional request. For example, in the case of trauma, the provisional request unit applies an algorithm that makes provisional requests based on the degree of bleeding and wound depth. In the case of internal diseases, the provisional request unit applies an algorithm that makes provisional requests based on the details of symptoms. Furthermore, in the case of pet illnesses or injuries, the provisional request unit applies an algorithm that makes provisional requests based on animal-specific symptoms. Thus, by applying different provisional request algorithms according to the category of illness or injury, the provisional request unit enables rapid and appropriate response. Specifically, the provisional request unit comprises multiple hardware and software elements such as a category determination module, an algorithm selection module, and a hospital arrangement engine. The category determination module receives image data (e.g., trauma images, tumor images, animal images), text data (e.g., “fall,” “fever,” “loss of appetite,” etc.), and audio data from the analysis unit or reception unit, and uses natural language processing models and image classification neural networks (e.g., ResNet, VGG, etc.) to determine category labels (e.g., “trauma,” “internal disease,” “animal disease,” etc.). Examples of AI input include “Image: bleeding image, text: ‘fell and bleeding’” and “Image: dog swelling image, text: ‘loss of appetite.’” AI output is structured data such as category labels (e.g., “trauma,” “animal disease”). The algorithm selection module receives the category determination result and automatically switches the provisional request algorithm of the hospital arrangement engine (e.g., trauma provisional request logic, internal disease provisional request logic, animal-specific symptom provisional request logic, etc.). For example, in the case of trauma, wound depth estimation and bleeding volume analysis are emphasized; in the case of internal disease, symptom progression and complication risk are emphasized; in the case of animal disease, species determination and extraction of specific symptoms are performed. Examples of AI output include “Trauma: detailed provisional request+wound depth explanation,” “Internal disease: observation provisional request+complication risk presented,” and “Animal disease: animal hospital provisional request+species explanation.” As subsequent processing, the provisional request result is sent to the interface control module and notification engine and used for decision support in medical settings and optimization of user experience. The technical effect is that the provisional request unit eliminates human empirical rules and manual algorithm selection, and achieves high-precision, high-speed category determination and automatic algorithm switching by AI, thereby enabling optimal provisional request for each case, reducing misjudgments, alleviating the burden on medical sites, and improving overall process efficiency. Specific application fields include emergency response in general households, triage in animal hospitals, remote medical support, and response to multiple casualties in disasters. Unlike conventional human visual inspection and fixed algorithm application, the present invention employs unconventional and technical methods such as AI inference in high-dimensional feature space, category-linked algorithm selection, and real-time provisional request optimization, thereby contributing to the improvement of computer technology itself.

The provisional request unit can estimate the user's emotion and adjust the display method of the provisional request result based on the estimated emotion. For example, if the user is feeling anxious, the provisional request unit provides detailed provisional request results to give reassurance. If the user is relaxed, the provisional request unit provides standard provisional request results. Furthermore, if the user is feeling rushed, the provisional request unit provides concise and rapid provisional request results. Thus, by adjusting the display method of the provisional request result according to the user's emotion, the provisional request unit can provide results that are easy for the user to understand. Emotion estimation is realized, for example, by using an emotion engine or generative AI with emotion estimation functions. Generative AI may include text generation AI (e.g., LLM) or multimodal generative AI, but is not limited to these examples. Specifically, the provisional request unit comprises multiple hardware and software elements such as an emotion estimation module, a display control module, and a hospital arrangement engine. The emotion estimation module receives user input data (e.g., text such as “What should I do, I'm worried,” audio data in WAV format 16 kHz, facial expression images, etc.) and uses natural language processing models, audio emotion recognition models, and image emotion estimation models to estimate the user's emotional state (e.g., anxiety, urgency, relaxation, etc.). Examples of AI input include “Text: ‘Please help me,’” “Audio: trembling voice,” and “Image: anxious facial expression.” AI output is structured data such as emotion labels (e.g., anxiety, urgency, relaxation), emotion scores (e.g., anxiety level 0.85, etc.), and confidence (e.g., 0.92). The display control module receives the emotion estimation result and automatically switches the display method of the provisional request result (e.g., detailed report display, simple summary display, reassurance message addition, color tone/layout change, etc.). For example, if anxiety is estimated, detailed explanations and reassurance messages (e.g., “Please stay calm. The provisional request to the hospital has been completed.”) are added to the provisional request result; for relaxation, standard summary display is applied; for urgency, only key points are emphasized in a concise manner. Examples of AI output include “Detailed provisional request report+reassurance message,” “Standard summary,” and “Concise key point display.” As subsequent processing, the display control result is sent to the user interface and notification engine and used for optimizing user experience and input assistance. The technical effect is that the provisional request unit does not depend on human subjective observation or empirical rules, and achieves high-precision, high-speed emotion estimation and display control by AI, thereby enabling optimal information presentation according to the user's psychological state, preventing misunderstandings, and improving satisfaction. Specific application fields include emergency response in general households, triage in animal hospitals, remote medical support, response to multiple casualties in disasters, and medical settings requiring psychological care. Unlike conventional human observation and fixed display, the present invention employs unconventional and technical methods such as AI inference in high-dimensional feature space, multimodal emotion estimation, and real-time UI optimization, thereby contributing to the improvement of computer technology itself.

The provisional request unit can determine the priority of provisional request based on the time of occurrence of an illness or injury during provisional request. For example, in the case of a recently occurred illness or injury, the provisional request unit performs prioritized provisional request and prompts rapid response. In the case of an illness or injury that occurred in the past, the provisional request unit can perform standard provisional request. Furthermore, the provisional request unit can adjust the priority of provisional request according to the time of occurrence of the illness or injury and prompt appropriate response. By determining the priority of provisional request according to the time of occurrence of the illness or injury, the provisional request unit enables prompt response. Specifically, the provisional request unit comprises multiple hardware and software elements such as an occurrence time determination module, a priority control module, and a hospital arrangement engine. The occurrence time determination module analyzes input data received from the analysis unit or reception unit (e.g., image data capture time, text such as “fever since this morning,” “fell yesterday,” audio data timestamps) and determines the time of occurrence (e.g., within the last hour, within 24 hours, one week ago, etc.). Examples of AI input include “Image: bleeding image (capture time: current time−30 minutes), text: ‘pain since this morning’” and “Image: swelling image (capture time: 2 days ago), text: ‘fell 2 days ago.’” AI output is structured data such as occurrence time labels (e.g., “recent,” “past”) and priority scores (e.g., 0.95, 0.45). The priority control module receives the occurrence time determination result and automatically adjusts the provisional request order and resource allocation of the hospital arrangement engine (e.g., prioritized provisional request for recent cases, batch processing for past cases). For example, immediate provisional request mode is selected for recent occurrences, and standard provisional request mode is applied for past cases. Examples of AI output include “recent occurrence: immediate provisional request, rapid result” and “past occurrence: standard provisional request, detailed report.” As subsequent processing, the priority control result is sent to the interface control module and notification engine and used for decision support in medical settings and optimization of user experience. The technical effect is that the provisional request unit eliminates human memory and manual chronological management, and achieves high-precision, high-speed occurrence time determination and priority control by AI, thereby enabling rapid response to emergency cases, reducing misjudgments, alleviating the burden on medical sites, and improving overall process efficiency. Specific application fields include emergency response in general households, triage in animal hospitals, remote medical support, and response to multiple casualties in disasters. Unlike conventional human chronological management and fixed-order provisional request, the present invention employs unconventional and technical methods such as AI inference in high-dimensional feature space, occurrence time-linked priority control, and real-time provisional request optimization, thereby contributing to the improvement of computer technology itself.

The provisional request unit can improve the accuracy of provisional request by referring to related literature on illnesses or injuries during provisional request. For example, the provisional request unit refers to the latest research papers on illnesses or injuries to improve the accuracy of provisional request. The provisional request unit can also refer to past case data to improve provisional request accuracy. Furthermore, the provisional request unit can optimize provisional request algorithms based on related literature on illnesses or injuries to further improve accuracy. Thus, by referring to related literature, the provisional request unit can enhance the accuracy of provisional request. Specifically, the provisional request unit comprises multiple hardware and software elements such as a literature search module, a case database linkage module, and an algorithm optimization module. The literature search module searches and retrieves the latest research papers and case reports related to symptoms or images extracted by the analysis unit from online medical literature databases and in-hospital case databases. Examples of AI input include “Text: ‘novel virus infection,’ ‘fever and cough’” and “Image: skin rash image.” AI output is structured data such as a list of related literature (e.g., paper title, abstract, publication year) and case data (e.g., past diagnosis results, treatment course, prognosis information). The algorithm optimization module reflects the obtained literature and case data in the parameters of the provisional request algorithm (e.g., thresholds, feature selection, weighting, etc.), and performs model retraining and dynamic optimization of inference logic. For example, when the latest treatment methods or diagnostic criteria are presented in papers, the provisional request unit automatically updates the algorithm to improve provisional request accuracy. Examples of AI output include “Related paper: ‘Skin symptoms of novel virus infection’” and “Case data: diagnosis results of 100 past cases.” As subsequent processing, the results of literature and case data reference are sent to the interface control module and medical staff notification engine and used for improving diagnostic accuracy and supporting treatment policy decisions. The technical effect is that the provisional request unit eliminates human memory and manual literature reference, and achieves high-precision, high-speed literature search and algorithm optimization by AI, thereby enabling rapid reflection of the latest knowledge, reducing misjudgments, alleviating the burden on medical sites, and improving overall process efficiency. Specific application fields include emergency response in general households, triage in animal hospitals, remote medical support, response to multiple casualties in disasters, and support for diagnosis of rare diseases. Unlike conventional human literature reference and fixed algorithm application, the present invention employs unconventional and technical methods such as AI inference in high-dimensional feature space, literature-linked algorithm optimization, and real-time knowledge updating, thereby contributing to the improvement of computer technology itself.

The system according to the embodiment is not limited to the examples described above, and various modifications are possible, for example, as follows. Specifically, the present system allows for a wide variety of variations in hardware and software architecture of each component, types of AI models, data flow, input/output interfaces, communication protocols, user interface design, security mechanisms, addition of extension modules, and so on. The system can employ convolutional neural networks or Vision Transformers as image analysis neural networks, Transformer-based large language models or BERT-series models as natural language processing models, and hybrid configurations of convolutional neural networks and recurrent neural networks as speech analysis models. Furthermore, the system can flexibly change its configuration according to the implementation environment, such as distributed inference platforms on the cloud, lightweight models on edge devices, or parallel computing clusters using GPUs. In addition, the system can select various communication methods for data linkage among the reception unit, analysis unit, determination unit, request unit, and provisional request unit, such as REST API, gRPC, message queue, batch transfer, and so on. The input data formats for AI can also support various data types, including images (JPEG, PNG, DICOM, etc.), audio (WAV, MP3, PCM, etc.), text (UTF-8, JSON, XML, etc.), and time-series data (sensor logs, vital sign arrays, etc.). The output of AI can be flexibly designed according to the application, such as scores, labels, probability distributions, structured reports, time-series predictions, and so on. As post-processing, the analysis, determination, and request results can be linked to decision support systems in medical settings, electronic medical records, telemedicine platforms, mobile applications for patients, and so on. As a technical effect, the present system eliminates conventional fixed workflows and human-dependent determination and notification processes, and achieves high-precision, high-speed multimodal analysis by AI, real-time decision support, and flexible system scalability, thereby reducing the burden on medical sites, decreasing misjudgments, improving patient satisfaction, and streamlining the overall process. Specific application fields include emergency response in general households, triage in animal hospitals, telemedicine support, response to multiple casualties during disasters, medical sites requiring mental care, industrial accident response in workplaces, and emergency response in schools and public facilities. The present invention contributes to the improvement of computer technology itself by employing technical methods such as AI inference in high-dimensional feature spaces, unconventional real-time control, and multimodal data integration, which do not rely on conventional human visual inspection, empirical rules, or fixed flows.

The reception unit can also search for similar past cases based on the user's input and provide reference information. For example, based on symptoms or injury situations input by the user, the system displays the treatment progress and outcomes of patients with similar symptoms in the past. Additionally, the reception unit can provide information on general treatment methods and prognosis based on statistical data obtained from past cases. Furthermore, the reception unit can present appropriate questions to the user based on past cases to collect more detailed information. By searching for similar past cases and providing reference information based on the user's input, the reception unit can alleviate user anxiety and support appropriate responses. Specifically, the reception unit comprises multiple hardware and software elements such as a case search module, statistical information generation module, and question generation module. The case search module vectorizes user input data (e.g., image data, text such as “cut my foot,” “fever,” audio data) and performs similarity searches (e.g., cosine similarity, Euclidean distance) in a high-dimensional feature space within the case database. Examples of AI input include “Image: abrasion image, Text: ‘fell and bleeding’” and “Image: rash image, Text: ‘itching.’” AI output is structured data such as a list of similar cases (e.g., case ID, treatment progress, prognosis, treatment method, occurrence time), statistical information (e.g., cure rate, average treatment period, recurrence rate), and recommended question lists (e.g., “How severe is the pain?” “When did symptoms start?”). The statistical information generation module automatically aggregates results and prognosis distributions by treatment method from the retrieved case group and presents them in an easy-to-understand format for the user. The question generation module analyzes feature distributions and missing information in the case database to automatically generate additional information to be obtained. As post-processing, the reception unit sends this information to the analysis unit and determination unit, contributing to improved diagnostic accuracy and optimized user experience. As a technical effect, the reception unit achieves high-precision, high-speed similar case search, statistical information generation, and automatic question generation by AI, without relying on human memory or empirical rules, thereby alleviating user anxiety, improving information collection efficiency, reducing misjudgments, and reducing the burden on medical sites. Specific application fields include emergency response in general households, telemedicine support, triage in animal hospitals, response to multiple casualties during disasters, and support for rare disease diagnosis. Unlike conventional human case searches and empirical question presentation, the present invention contributes to the improvement of computer technology itself by employing unconventional and technical methods such as AI inference in high-dimensional feature spaces, case database-linked information presentation, and real-time question generation.

The analysis unit can also provide a function for real-time consultation with medical professionals based on the user's input. For example, the analysis unit analyzes symptoms or injury situations input by the user and, if necessary, prompts consultation with medical professionals. The analysis unit can also provide real-time feedback from medical professionals to the user, supporting appropriate responses. Furthermore, the analysis unit can record the content of consultations with medical professionals and save it as reference information for the future. By providing a function for real-time consultation with medical professionals based on the user's input, the analysis unit can support prompt and appropriate responses. Specifically, the analysis unit comprises multiple hardware and software elements such as an expert collaboration module, feedback management module, and consultation record module. The expert collaboration module automatically determines whether consultation with a medical professional is necessary based on initial AI analysis results (e.g., image analysis scores, symptom estimation labels, severity scores) using threshold judgment or rule-based branching (e.g., severity above 0.7, unknown cases). Examples of AI input include “Image: swelling image, Text: ‘fever and swelling’” and “Image: bleeding image, Text: ‘cannot stop bleeding.’” AI output is structured data such as consultation necessity flags (e.g., required, not required), expert selection candidates (e.g., surgeon, internist), and consultation content templates (e.g., symptom summary, image attachment). The feedback management module receives responses from medical professionals (e.g., text comments, diagnosis labels, recommended treatments) in real time and performs post-processing such as summarization, translation, and importance assignment for the user. The consultation record module saves consultation history (e.g., consultation time, content, expert ID, feedback content) in a time-series database for future case analysis and AI model retraining. As post-processing, the analysis unit links expert feedback to the determination unit and request unit for decision support and emergency response optimization. As a technical effect, the analysis unit eliminates manual expert collaboration and record management by humans, and achieves high-precision, high-speed consultation necessity determination, real-time feedback management, and database creation by AI, thereby enabling rapid response to emergency cases, reducing misjudgments, reducing the burden on medical sites, and streamlining the overall process. Specific application fields include emergency response in general households, telemedicine support, triage in animal hospitals, response to multiple casualties during disasters, and diagnostic support in areas with a shortage of specialists. Unlike conventional sequential human contact and record management, the present invention contributes to the improvement of computer technology itself by employing unconventional and technical methods such as AI inference and automated expert collaboration, and real-time data integration.

The determination unit can estimate the user's emotion and adjust the explanation method when determining the necessity of medical consultation based on the estimated emotion. For example, if the user feels anxious, the system provides a detailed explanation to reassure the user. If the user is relaxed, a concise explanation is provided. Furthermore, if the user is agitated, a prompt explanation is provided to encourage rapid response. By adjusting the explanation method according to the user's emotion when determining the necessity of medical consultation, the determination unit can provide explanations that are easy for the user to understand. Specifically, the determination unit comprises multiple hardware and software elements such as an emotion estimation module, explanation generation module, and severity determination engine. The emotion estimation module receives user input data (e.g., text such as “What should I do? I'm worried,” audio data, facial expression images) and estimates the emotional state (e.g., anxiety, agitation, relaxation) using natural language processing models, speech emotion recognition models, and image emotion estimation models. Examples of AI input include “Text: ‘Please help,’” “Audio: trembling voice,” “Image: anxious facial expression.” AI output is structured data such as emotion labels (e.g., anxiety, agitation, relaxation), emotion scores (e.g., anxiety level 0.85), and confidence (e.g., 0.92). The explanation generation module receives emotion estimation results and severity determination results, and uses explanation text generation AI (e.g., large language models) to automatically adjust the level of detail, tone, and message content (e.g., reassuring sentences, concise key points, prompt instructions). For example, in the case of anxiety: “Please stay calm. I will explain the reasons for medical consultation and the next steps in detail.” In the case of relaxation: “I will briefly explain only the standard reasons for medical consultation.” In the case of agitation: “Urgent medical consultation is necessary. I will emphasize only the key points.” Examples of AI output include “Detailed explanation+reassurance message,” “Simple explanation,” “Key point emphasis explanation.” As post-processing, the explanation generation results are sent to the user interface or notification engine and used to optimize user experience and prevent misunderstandings. As a technical effect, the determination unit achieves high-precision, high-speed emotion estimation and explanation generation control by AI, without relying on subjective human explanations or empirical rules, thereby providing optimal information presentation, preventing misunderstandings, and improving satisfaction according to the user's psychological state. Specific application fields include emergency response in general households, triage in animal hospitals, telemedicine support, response to multiple casualties during disasters, and medical sites requiring mental care. Unlike conventional human explanations or fixed explanation patterns, the present invention contributes to the improvement of computer technology itself by employing unconventional and technical methods such as AI inference in high-dimensional feature spaces, emotion-linked explanation generation, and real-time UI optimization.

The request unit can estimate the user's emotion and adjust the level of detail of information provided when requesting an ambulance based on the estimated emotion. For example, if the user feels anxious, the system provides detailed information to reassure the user. If the user is relaxed, standard information is provided. Furthermore, if the user is agitated, concise and prompt information is provided to encourage rapid response. By adjusting the level of detail of information provided when requesting an ambulance according to the user's emotion, the request unit enables rapid and appropriate response. Specifically, the request unit comprises multiple hardware and software elements such as an emotion estimation module, information detail control module, and ambulance dispatch engine. The emotion estimation module receives user input data (e.g., text such as “What should I do? I'm worried,” audio data, facial expression images) and estimates the emotional state (e.g., anxiety, agitation, relaxation) using natural language processing models, speech emotion recognition models, and image emotion estimation models. Examples of AI input include “Text: ‘Please help,’” “Audio: trembling voice,” “Image: anxious facial expression.” AI output is structured data such as emotion labels (e.g., anxiety, agitation, relaxation), emotion scores (e.g., anxiety level 0.85), and confidence (e.g., 0.92). The information detail control module receives emotion estimation results and dynamically adjusts the level of detail of request information in the ambulance dispatch engine (e.g., patient condition, vital signs, candidate hospitals for transport, progress notification frequency). For example, in the case of anxiety, detailed patient information, reassurance messages, and progress notifications are provided; in the case of relaxation, only standard information is provided; in the case of agitation, the minimum necessary information is used for immediate request. Examples of AI output include “Detailed request+reassurance message,” “Standard request,” “Simple request.” As post-processing, the information detail control results are sent to the interface control module or notification engine and used to optimize user experience and support decision-making in medical settings. As a technical effect, the request unit achieves high-precision, high-speed emotion estimation and information detail control by AI, without relying on subjective human judgment or empirical rules, thereby providing optimal ambulance requests, preventing misunderstandings, and improving satisfaction according to the user's psychological state. Specific application fields include emergency response in general households, triage in animal hospitals, telemedicine support, response to multiple casualties during disasters, and medical sites requiring mental care. Unlike conventional human information presentation or fixed request flows, the present invention contributes to the improvement of computer technology itself by employing unconventional and technical methods such as AI inference in high-dimensional feature spaces, emotion-linked information control, and real-time request optimization.

The provisional request unit can estimate the user's emotion and adjust the priority of information provided when making a provisional request to a hospital based on the estimated emotion. For example, if the user feels anxious, the system prioritizes important information to reassure the user. If the user is relaxed, standard information is provided. Furthermore, if the user is agitated, the most important information is provided promptly to encourage rapid response. By adjusting the priority of information provided when making a provisional request to a hospital according to the user's emotion, the provisional request unit enables rapid and appropriate response. Specifically, the provisional request unit comprises multiple hardware and software elements such as an emotion estimation module, information priority control module, and hospital arrangement engine. The emotion estimation module receives user input data (e.g., text such as “I'm very anxious,” audio data, facial expression images) and estimates the emotional state (e.g., anxiety, agitation, relaxation) using natural language processing models, speech emotion recognition models, and image emotion estimation models. Examples of AI input include “Text: ‘What should I do? I'm worried,’” “Audio: trembling voice saying ‘Please help,’” “Image: facial expression with furrowed brows.” AI output is structured data such as emotion labels (e.g., anxiety, agitation, relaxation), emotion scores (e.g., anxiety level 0.85), and confidence (e.g., 0.92). The information priority control module receives emotion estimation results and dynamically adjusts the priority of provisional request information in the hospital arrangement engine (e.g., patient condition, vital signs, candidate hospitals for transport, progress notifications). For example, in the case of anxiety, important information is presented with highest priority; in the case of relaxation, standard order is used; in the case of agitation, only the most important information is sent immediately. Examples of AI output include “Important information prioritized+reassurance message,” “Standard information order,” “Immediate transmission of most important information.” As post-processing, the information priority control results are sent to the interface control module or notification engine and used to optimize user experience and support decision-making in medical settings. As a technical effect, the provisional request unit achieves high-precision, high-speed emotion estimation and information priority control by AI, without relying on subjective human judgment or empirical rules, thereby providing optimal provisional hospital requests, preventing misunderstandings, and improving satisfaction according to the user's psychological state. Specific application fields include emergency response in general households, triage in animal hospitals, telemedicine support, response to multiple casualties during disasters, and medical sites requiring mental care. Unlike conventional human information presentation or fixed provisional request flows, the present invention contributes to the improvement of computer technology itself by employing unconventional and technical methods such as AI inference in high-dimensional feature spaces, emotion-linked information control, and real-time provisional request optimization.

The reception unit can also provide relevant health information and preventive measures based on the user's input. For example, based on symptoms or injury situations input by the user, the system displays relevant health information and preventive measures. Additionally, the reception unit can provide health management advice and lifestyle improvement suggestions based on the user's input. Furthermore, the reception unit can set reminders for regular health checks based on the user's input. By providing relevant health information and preventive measures based on the user's input, the reception unit can support the user's health management. Specifically, the reception unit comprises multiple hardware and software elements such as a health information search module, preventive measure proposal module, and reminder generation module. The health information search module analyzes user input data (e.g., symptom text, images, audio, etc.) and automatically extracts relevant health information (e.g., disease explanations, lifestyle disease risks, infectious disease prevention methods) using natural language processing models and image classification models. Examples of AI input include “Text: ‘persistent cough,’” “Image: skin rash image.” AI output is structured data such as health information lists (e.g., disease explanations, preventive measures, lifestyle improvement suggestions) and reminder setting information (e.g., regular checkup dates, vaccination dates). The preventive measure proposal module automatically generates individualized preventive measures (e.g., encouragement of hand washing, exercise habits, dietary improvements) according to symptoms and risk factors. The reminder generation module automatically sets notifications for regular health checks and follow-ups based on the user's health condition and past history. As post-processing, the reception unit links this information to the user interface and analysis unit, contributing to health management support and promotion of preventive medicine. As a technical effect, the reception unit achieves high-precision, high-speed health information extraction, preventive measure proposal, and automatic reminder generation by AI, without relying on human knowledge or empirical rules, thereby improving health management efficiency, promoting preventive medicine, and reducing the burden on medical sites. Specific application fields include health management in general households, telemedicine support, health guidance in schools and workplaces, and preventive awareness during infectious disease outbreaks. Unlike conventional human information provision or manual reminder setting, the present invention contributes to the improvement of computer technology itself by employing unconventional and technical methods such as AI inference and health information-linked control, and real-time notifications.

The analysis unit can also predict the progression of symptoms or injuries based on the user's input and propose appropriate responses. For example, the analysis unit analyzes symptoms or injury situations input by the user and predicts the progression of symptoms or injuries. Additionally, the analysis unit can propose appropriate countermeasures or treatment methods based on the prediction results. Furthermore, the analysis unit can propose regular follow-ups to the user according to the progression of symptoms or injuries. By predicting the progression of symptoms or injuries based on the user's input and proposing appropriate responses, the analysis unit can support the user's health management. Specifically, the analysis unit comprises multiple hardware and software elements such as a progression prediction module, countermeasure proposal module, and follow-up proposal module. The progression prediction module takes user input data (e.g., image data, symptom text, time-series vital sign data) as input and uses time-series analysis models (e.g., LSTM, GRU, Transformer-based time-series models) and image analysis neural networks to calculate progression predictions (e.g., probability of worsening, predicted days to recovery, risk of complications). Examples of AI input include “Image: swelling image+past 3 days of progress images, Text: ‘swelling is spreading,’” “Time-series vital signs: fever trend.” AI output is structured data such as progression prediction scores (e.g., probability of worsening 0.72, predicted days to recovery 5), risk labels (e.g., high risk, low risk), and recommended countermeasures (e.g., recommendation to seek medical consultation, observation, first aid). The countermeasure proposal module automatically generates treatment methods, lifestyle guidance, and timing for medical consultation based on progression prediction results. The follow-up proposal module automatically sets regular re-evaluations, additional questions, reminders, etc., according to progression and risk. As post-processing, the analysis unit links this information to the determination unit and request unit, contributing to overall health management optimization and reduction of burden on medical sites. As a technical effect, the analysis unit eliminates empirical rules and manual observation by humans, and achieves high-precision, high-speed progression prediction, countermeasure proposal, and follow-up automation by AI, thereby reducing misjudgments, enabling early response, and improving health management efficiency. Specific application fields include health management in general households, observation of chronic disease progression, telemedicine support, and response to multiple casualties during disasters. Unlike conventional human observation or fixed countermeasure presentation, the present invention contributes to the improvement of computer technology itself by employing unconventional and technical methods such as AI inference and time-series data-linked control, and real-time progression prediction.

The determination unit can also consider the user's living environment and occupation when determining the necessity of medical consultation based on the user's input. For example, in addition to symptoms or injury situations input by the user, the determination unit considers the user's living environment and occupation to determine the necessity of medical consultation. Additionally, the determination unit can propose appropriate medical institutions or specialists based on the user's living environment and occupation. Furthermore, the determination unit can propose follow-up or rehabilitation after medical consultation according to the user's living environment and occupation. By considering the user's living environment and occupation, the determination unit can determine the necessity of medical consultation more appropriately and support the user's health management. Specifically, the determination unit comprises multiple hardware and software elements such as an environment/occupation information analysis module, medical institution proposal module, and follow-up proposal module. The environment/occupation information analysis module vectorizes user input data (e.g., occupation “construction worker,” living environment “hot and humid,” “night shift”) using natural language processing models and combines it with symptoms and risk factors for input to the medical consultation necessity determination engine. Examples of AI input include “Text: ‘back pain,’ ‘job involving heavy lifting,’” “Text: ‘fever,’ ‘working at a senior care facility.’” AI output is structured data such as medical consultation recommendation scores (e.g., 0.88), recommended medical institution lists (e.g., orthopedic surgery, infectious disease specialist), and follow-up proposals (e.g., rehabilitation, regular checkups). The medical institution proposal module automatically selects optimal medical institutions or specialists according to occupation, living environment, and symptoms. The follow-up proposal module automatically generates post-consultation lifestyle guidance, rehabilitation plans, and re-examination reminders. As post-processing, the determination unit links this information to the request unit and user interface, contributing to health management optimization and reduction of burden on medical sites. As a technical effect, the determination unit eliminates empirical rules and manual selection of medical institutions and follow-up proposals by humans, and achieves high-precision, high-speed environment/occupation information analysis and individualized proposals by AI, thereby reducing misjudgments, guiding optimal medical consultation, and improving health management efficiency. Specific application fields include industrial accident response in workplaces, health management in schools and senior care facilities, telemedicine support, and follow-up for chronic diseases. Unlike conventional human judgment or fixed proposals, the present invention contributes to the improvement of computer technology itself by employing unconventional and technical methods such as AI inference and individualized information-linked control, and real-time medical institution selection.

The request unit can also customize the information provided when requesting an ambulance based on the user's input. For example, the request unit customizes the information provided when requesting an ambulance based on symptoms or injury situations input by the user. Additionally, the request unit can arrange necessary medical equipment and medical staff for ambulance dispatch based on the user's input. Furthermore, the request unit can provide information on the expected arrival time of the ambulance and the hospital for patient transport based on the user's input. By customizing the information provided when requesting an ambulance based on the user's input, the request unit enables rapid and appropriate response. Specifically, the request unit comprises multiple hardware and software elements such as an information customization module, medical resource arrangement module, and arrival prediction module. The information customization module analyzes user input data (e.g., images, symptom text, vital signs) and automatically extracts and formats necessary information for ambulance requests (e.g., patient condition, medical history, allergy information). Examples of AI input include “Image: bleeding image, Text: ‘consciousness disorder,’” “Image: swelling image, Text: ‘difficulty breathing.’” AI output is structured data such as request information sets (e.g., patient condition, necessary medical equipment, recommended staff composition, expected arrival time, candidate hospitals for transport). The medical resource arrangement module automatically selects and arranges necessary medical equipment (e.g., AED, oxygen cylinder) and staff (e.g., emergency medical technicians, nurses) according to symptoms and severity. The arrival prediction module uses user location information and traffic data to calculate the expected arrival time of the ambulance and optimal hospital for transport in real time. As post-processing, the request unit links this information to the interface control module and notification engine, contributing to decision support in medical settings and optimization of user experience. As a technical effect, the request unit eliminates empirical rules and manual information formatting and resource arrangement by humans, and achieves high-precision, high-speed information customization, resource optimization, and arrival prediction by AI, thereby reducing misjudgments, enabling rapid response, and reducing the burden on medical sites. Specific application fields include emergency response in general households, telemedicine support, triage in animal hospitals, and response to multiple casualties during disasters. Unlike conventional human information presentation or manual arrangement, the present invention contributes to the improvement of computer technology itself by employing unconventional and technical methods such as AI inference and information-linked control, and real-time resource optimization.

The provisional request unit can also customize the information provided when making a provisional request to a hospital based on the user's input. For example, the provisional request unit customizes the information provided when making a provisional request to a hospital based on symptoms or injury situations input by the user. Additionally, the provisional request unit can arrange necessary medical equipment and medical staff for hospital admission based on the user's input. Furthermore, the provisional request unit can notify the hospital of the expected arrival time and the patient's condition based on the user's input. By customizing the information provided when making a provisional request to a hospital based on the user's input, the provisional request unit enables rapid and appropriate response. Specifically, the provisional request unit comprises multiple hardware and software elements such as an information customization module, medical resource arrangement module, and arrival prediction module. The information customization module analyzes user input data (e.g., images, symptom text, vital signs) and automatically extracts and formats necessary information for provisional hospital requests (e.g., patient condition, medical history, allergy information). Examples of AI input include “Image: bleeding image, Text: ‘consciousness disorder,’” “Image: swelling image, Text: ‘difficulty breathing.’” AI output is structured data such as provisional request information sets (e.g., patient condition, necessary medical equipment, recommended staff composition, expected arrival time). The medical resource arrangement module automatically selects and arranges necessary medical equipment and staff according to symptoms and severity. The arrival prediction module uses user location information and traffic data to calculate the expected arrival time at the hospital in real time. As post-processing, the provisional request unit links this information to the interface control module and notification engine, contributing to decision support in medical settings and optimization of user experience. As a technical effect, the provisional request unit eliminates empirical rules and manual information formatting and resource arrangement by humans, and achieves high-precision, high-speed information customization, resource optimization, and arrival prediction by AI, thereby reducing misjudgments, enabling rapid response, and reducing the burden on medical sites. Specific application fields include emergency response in general households, telemedicine support, triage in animal hospitals, and response to multiple casualties during disasters. Unlike conventional human information presentation or manual arrangement, the present invention contributes to the improvement of computer technology itself by employing unconventional and technical methods such as AI inference and information-linked control, and real-time resource optimization.

The following is a brief explanation of the processing flow of Example of the Embodiment. Specifically, the present system automates and optimizes the entire process from user input to emergency response and hospital coordination by linking each module—reception unit, analysis unit, determination unit, request unit, and provisional request unit—and utilizing multimodal AI models and real-time data flow. The reception unit receives various data such as images, audio, text, and location information, and performs AI-based preprocessing (e.g., image noise removal, audio spectrum conversion, text normalization). The analysis unit uses image analysis neural networks (e.g., convolutional neural networks, Vision Transformers), natural language processing models (e.g., Transformer-based large language models), and speech analysis models (e.g., CNN+RNN hybrid) to infer severity scores, symptom labels, progression predictions, and emotional states in high-dimensional feature spaces. The determination unit integrates analysis results with user attributes (e.g., living environment, occupation) and emotion estimation results to individualize and automate the necessity of medical consultation, recommended medical institutions, and explanation text generation. The request unit optimizes in real time the level of detail, content, resource arrangement, and arrival prediction of ambulance request information based on the output of the determination unit, and the provisional request unit automatically adjusts the priority, content, resource arrangement, and arrival prediction of provisional hospital request information. Examples of AI input/output for each unit include “Input: image (bleeding image), text (‘severe pain’), audio (distressed voice)→Output: severity 0.88, symptom label ‘bleeding,’ emotion ‘anxiety’”; “Input: severity 0.88, emotion ‘anxiety’→Output: medical consultation recommended+detailed explanation”; “Input: medical consultation recommended→Output: ambulance request (with detailed information, arrival prediction 5 minutes).” As post-processing, the output of each unit is linked to the user interface, decision support systems in medical settings, electronic medical records, notification engines, and so on. As a technical effect, the present system eliminates conventional human-dependent sequential processing and fixed flows, and achieves high-precision, high-speed multimodal analysis, individualized decision-making, and real-time resource optimization by AI, thereby reducing misjudgments, enabling rapid response, reducing the burden on medical sites, improving patient satisfaction, and streamlining the overall process. Specific application fields include emergency response in general households, triage in animal hospitals, telemedicine support, response to multiple casualties during disasters, and medical sites requiring mental care. Unlike conventional human sequential judgment or fixed flows, the present invention contributes to the improvement of computer technology itself by employing unconventional and technical methods such as AI inference in high-dimensional feature spaces, multimodal data integration, and real-time decision optimization.

Step 1: The reception unit receives information from the user. The information from the user includes photographs of illnesses or injuries, descriptions of symptoms, location information, and so on. For example, the reception unit receives photographs of illnesses or injuries taken by the user with a smartphone, text information or audio information input by the user. The reception unit can also obtain the user's location information and use it for ambulance arrangement. Step 2: The analysis unit analyzes the information received by the reception unit. For example, the analysis unit uses image analysis algorithms to analyze photographs of illnesses or injuries and assess the degree of bleeding or depth of wounds. The analysis unit also uses natural language processing technology to analyze text information and determine the condition of the illness or injury from the user's description of symptoms. Furthermore, the analysis unit uses speech analysis technology to analyze audio information and interpret the user's spoken description of symptoms. Step 3: The determination unit determines the necessity of medical consultation based on the information analyzed by the analysis unit. For example, the determination unit evaluates the severity and urgency of the illness or injury based on the analysis results and determines the necessity of medical consultation based on the degree of bleeding, depth of wounds, details of symptoms, and so on. Step 4: The request unit requests an ambulance when medical consultation is determined to be necessary. For example, the request unit arranges the nearest ambulance based on the user's location information and requests an ambulance using an emergency call system. Step 5: The provisional request unit makes a provisional request to a hospital for patient transport when an ambulance has been requested. For example, the provisional request unit notifies the hospital of the patient's condition and expected arrival time, and provides information such as details of symptoms, vital signs, and expected arrival time. This enables the hospital to prepare for acceptance and shorten transport time. Specifically, in Step 1, the reception unit receives image data (e.g., JPEG, PNG), audio data (e.g., WAV format 16 kHz), text data (e.g., UTF-8), location information (e.g., GPS coordinates), and so on, and performs AI-based preprocessing (e.g., image noise removal, audio spectrum conversion, text normalization). In Step 2, the analysis unit uses image analysis neural networks (e.g., convolutional neural networks, Vision Transformers), natural language processing models (e.g., Transformer-based large language models), and speech analysis models (e.g., CNN+RNN hybrid) to infer severity scores (e.g., 0.0-1.0), symptom labels (e.g., ‘bleeding,’ ‘swelling’), progression predictions (e.g., probability of worsening 0.72), and emotional states (e.g., anxiety, agitation) in high-dimensional feature spaces. Examples of AI input include “Image: bleeding image, Text: ‘severe pain,’ Audio: distressed voice,” “Image: swelling image, Text: ‘swelling is spreading.’” AI output is structured data such as severity scores, symptom labels, progression predictions, and emotion labels. In Step 3, the determination unit integrates analysis results with user attributes (e.g., living environment, occupation) and emotion estimation results to individualize and automate the necessity of medical consultation (e.g., medical consultation recommended, home care recommended), recommended medical institutions, and explanation text generation. Examples of AI output include “Severity 0.88, emotion ‘anxiety’→medical consultation recommended+detailed explanation,” “Severity 0.62, emotion ‘relaxation’→home first aid recommended.” In Step 4, the request unit optimizes in real time the level of detail, content, resource arrangement, and arrival prediction of ambulance request information (e.g., patient condition, necessary medical equipment, expected arrival time) based on the output of the determination unit. Examples of AI output include “Detailed request+reassurance message,” “Standard request,” “Simple request.” In Step 5, the provisional request unit automatically adjusts the priority, content, resource arrangement, and arrival prediction of provisional hospital request information (e.g., patient condition, vital signs, expected arrival time). Examples of AI output include “Important information prioritized+reassurance message,” “Standard information order,” “Immediate transmission of most important information.” As post-processing, the output of each unit is linked to the user interface, decision support systems in medical settings, electronic medical records, notification engines, and so on. As a technical effect, the present system eliminates conventional human-dependent sequential processing and fixed flows, and achieves high-precision, high-speed multimodal analysis, individualized decision-making, and real-time resource optimization by AI, thereby reducing misjudgments, enabling rapid response, reducing the burden on medical sites, improving patient satisfaction, and streamlining the overall process. Specific application fields include emergency response in general households, triage in animal hospitals, telemedicine support, response to multiple casualties during disasters, and medical sites requiring mental care. Unlike conventional human sequential judgment or fixed flows, the present invention contributes to the improvement of computer technology itself by employing unconventional and technical methods such as AI inference in high-dimensional feature spaces, multimodal data integration, and real-time decision optimization.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, determination unit, request unit, and provisional request unit is implemented by at least one of, for example, a smart device 14 and a data processing apparatus 12. For example, the reception unit is implemented by a control unit 46A of the smart device 14 and receives photographs of an illness or injury taken by the user with a smartphone and text information input by the user. The analysis unit is implemented, for example, by a specific processing unit 290 of the data processing apparatus 12 and analyzes the information using image analysis algorithms and natural language processing techniques. The determination unit is implemented, for example, by the specific processing unit 290 of the data processing apparatus 12 and determines the necessity of medical consultation based on the analysis result. The request unit is implemented, for example, by the control unit 46A of the smart device 14 and arranges the nearest ambulance based on the user's location information. The provisional request unit is implemented, for example, by the specific processing unit 290 of the data processing apparatus 12 and notifies the hospital of the patient's condition and expected arrival time. The correspondence between each unit and the device or control unit is not limited to the examples described above and various modifications are possible.

### Second Embodiment

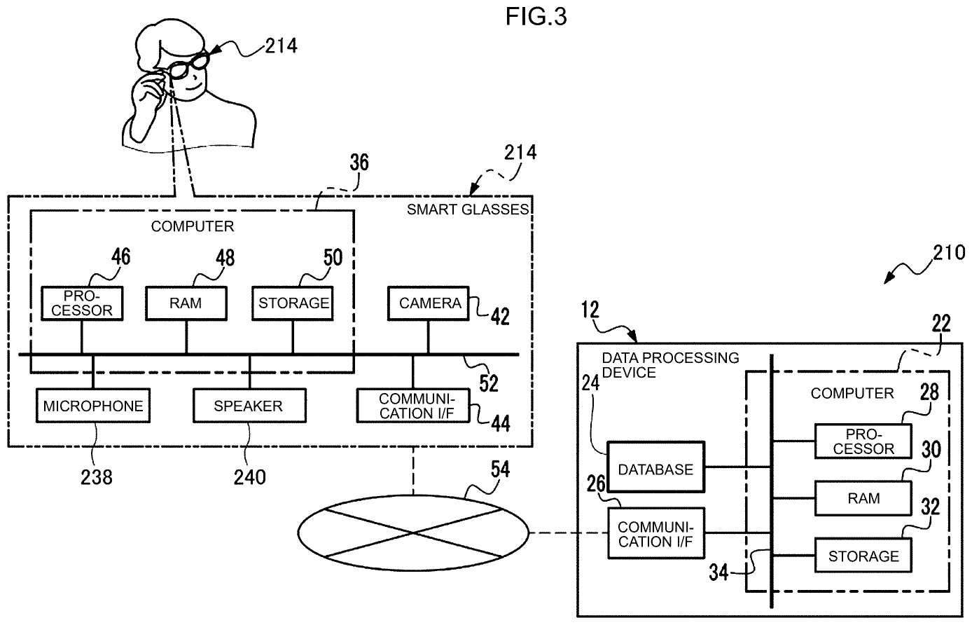

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

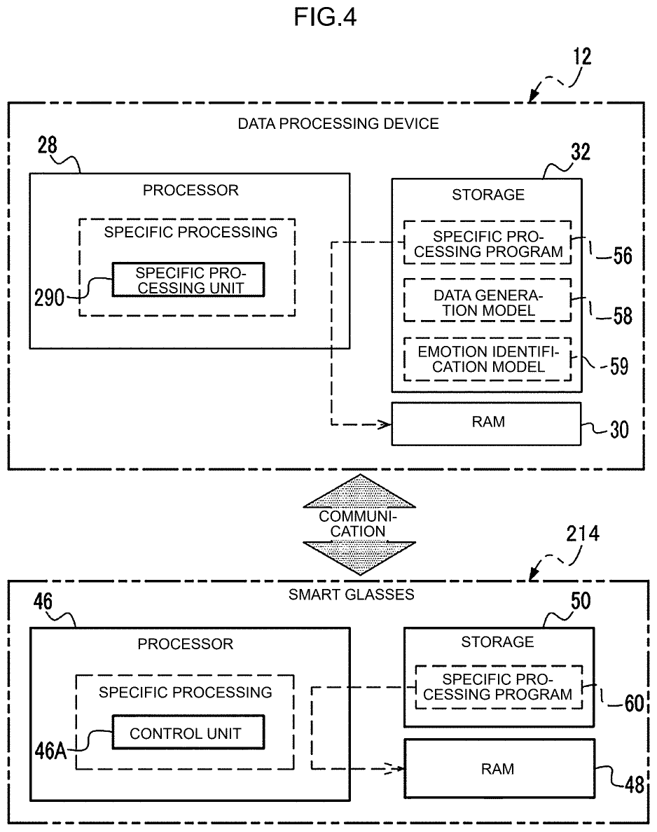

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, determination unit, request unit, and provisional request unit is implemented by at least one of, for example, smart glasses 214 and a data processing apparatus 12. For example, the reception unit is implemented by a control unit 46A of the smart glasses 214 and receives photographs of an illness or injury taken by the user with the smart glasses 214 and text information input by the user. The analysis unit is implemented, for example, by a specific processing unit 290 of the data processing apparatus 12 and analyzes the information using image analysis algorithms and natural language processing techniques. The determination unit is implemented, for example, by the specific processing unit 290 of the data processing apparatus 12 and determines the necessity of medical consultation based on the analysis result. The request unit is implemented, for example, by the control unit 46A of the smart glasses 214 and arranges the nearest ambulance based on the user's location information. The provisional request unit is implemented, for example, by the specific processing unit 290 of the data processing apparatus 12 and notifies the hospital of the patient's condition and expected arrival time. The correspondence between each unit and the device or control unit is not limited to the examples described above and various modifications are possible.

### Third Embodiment

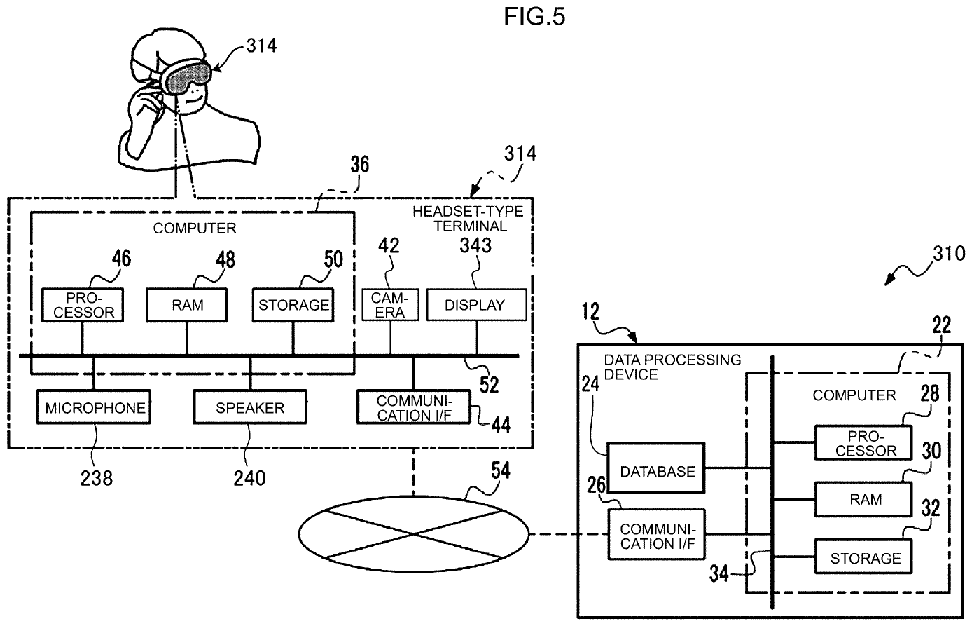

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

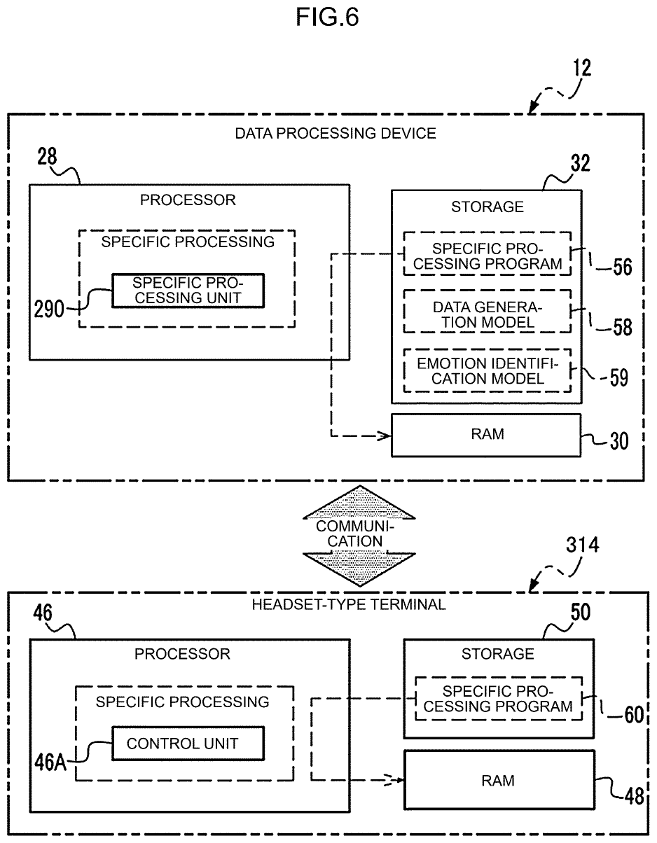

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, determination unit, request unit, and provisional request unit is implemented by at least one of, for example, a headset-type terminal 314 and a data processing apparatus 12. For example, the reception unit is implemented by a control unit 46A of the headset-type terminal 314 and receives photographs of an illness or injury taken by the user with the headset-type terminal 314 and text information input by the user. The analysis unit is implemented, for example, by a specific processing unit 290 of the data processing apparatus 12 and analyzes the information using image analysis algorithms and natural language processing techniques. The determination unit is implemented, for example, by the specific processing unit 290 of the data processing apparatus 12 and determines the necessity of medical consultation based on the analysis result. The request unit is implemented, for example, by the control unit 46A of the headset-type terminal 314 and arranges the nearest ambulance based on the user's location information. The provisional request unit is implemented, for example, by the specific processing unit 290 of the data processing apparatus 12 and notifies the hospital of the patient's condition and expected arrival time. The correspondence between each unit and the device or control unit is not limited to the examples described above and various modifications are possible.

### Fourth Embodiment

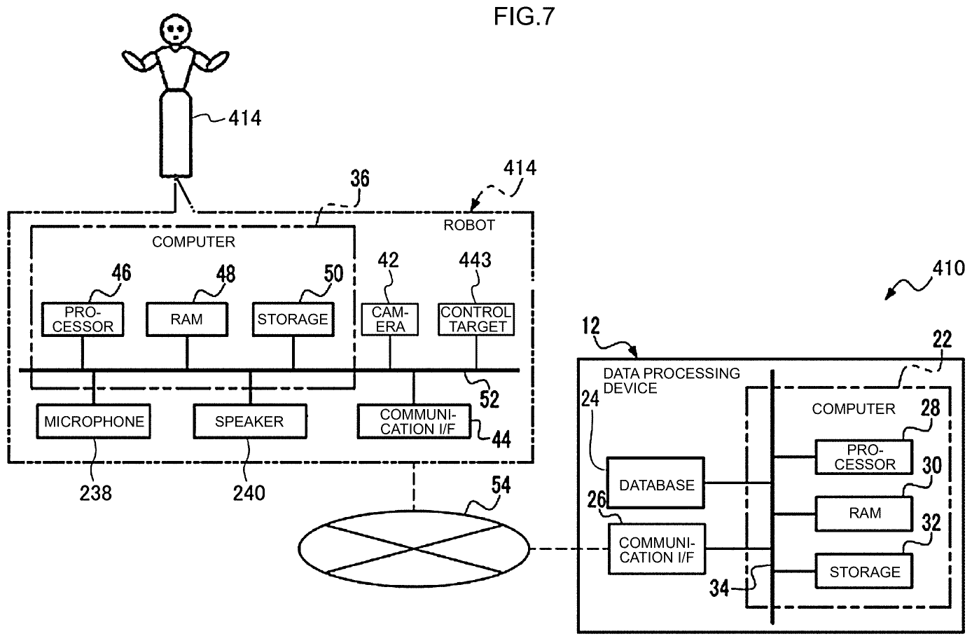

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

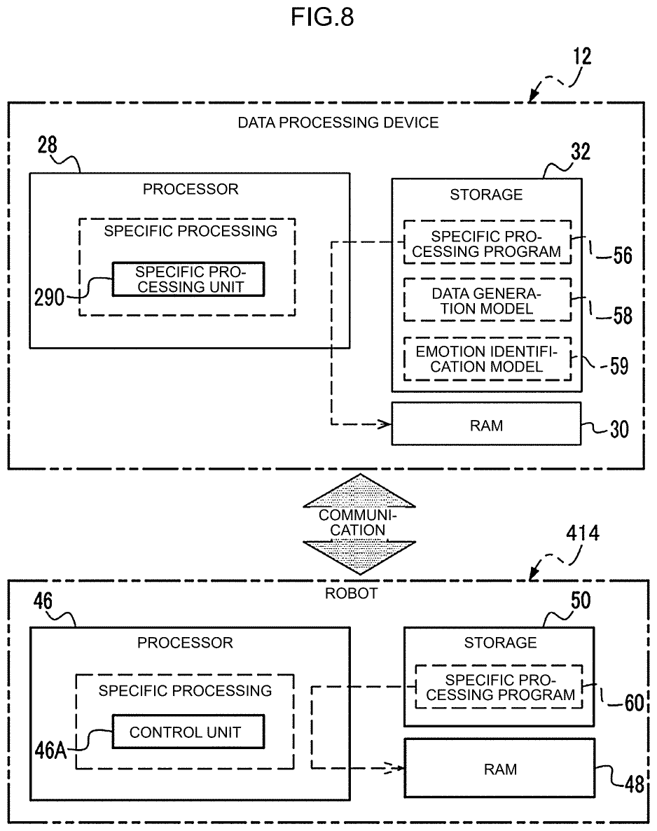

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, determination unit, request unit, and provisional request unit is implemented by at least one of, for example, a robot 414 and a data processing apparatus 12. For example, the reception unit is implemented by a control unit 46A of the robot 414 and receives photographs of an illness or injury taken by the user with the robot 414 and text information input by the user. The analysis unit is implemented, for example, by a specific processing unit 290 of the data processing apparatus 12 and analyzes the information using image analysis algorithms and natural language processing techniques. The determination unit is implemented, for example, by the specific processing unit 290 of the data processing apparatus 12 and determines the necessity of medical consultation based on the analysis result. The request unit is implemented, for example, by the control unit 46A of the robot 414 and arranges the nearest ambulance based on the user's location information. The provisional request unit is implemented, for example, by the specific processing unit 290 of the data processing apparatus 12 and notifies the hospital of the patient's condition and expected arrival time. The correspondence between each unit and the device or control unit is not limited to the examples described above and various modifications are possible.

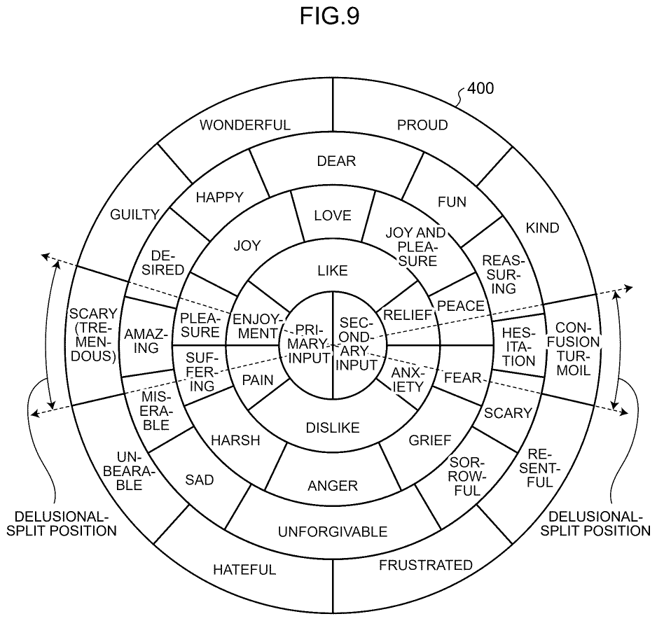

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

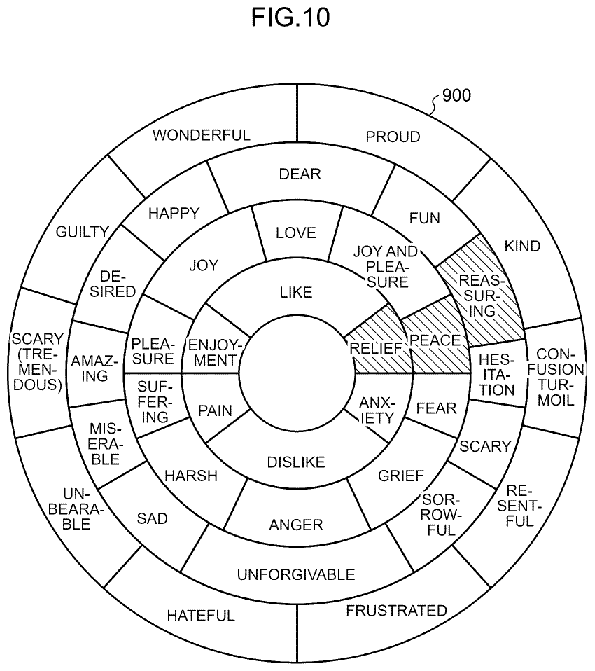

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1)A system comprising: a reception unit configured to receive information from a user; an analysis unit configured to analyze the information received by the reception unit; a determination unit configured to determine the necessity of medical consultation based on the information analyzed by the analysis unit; a request unit configured to request an ambulance when the determination unit determines that medical consultation is necessary; and a provisional request unit configured to make a provisional request to a hospital for patient transport when the request unit has requested an ambulance.
(Supplementary Note 2)The system according to Supplementary Note 1, wherein the reception unit is configured to receive a photograph or situation of an illness or injury input by the user.
(Supplementary Note 3)The system according to Supplementary Note 1, wherein the analysis unit is configured to analyze the received photograph or situation and determine the condition of the illness or injury.
(Supplementary Note 4)The system according to Supplementary Note 1, wherein the determination unit is configured to determine the necessity of medical consultation based on the analysis result.
(Supplementary Note 5)The system according to Supplementary Note 1, wherein the request unit is configured to request an ambulance when it is determined that medical consultation is necessary.
(Supplementary Note 6)The system according to Supplementary Note 1, wherein the provisional request unit is configured to make a provisional request to a hospital for patient transport when an ambulance has been requested.
(Supplementary Note 7)The system according to Supplementary Note 1, wherein the provisional request unit is configured to notify the hospital of the patient's condition or expected arrival time and prompt preparation of necessary medical staff or equipment.
(Supplementary Note 8)The system according to Supplementary Note 1, wherein the reception unit is configured to estimate the user's emotion and adjust the display method of the input interface based on the estimated emotion.
(Supplementary Note 9)The system according to Supplementary Note 1, wherein the reception unit is configured to analyze the user's past input history and propose an optimal input method.
(Supplementary Note 10)The system according to Supplementary Note 1, wherein the reception unit is configured to customize input items for entering photographs or situations of illness or injury based on the user's current health condition and past medical history.
(Supplementary Note 11)The system according to Supplementary Note 1, wherein the reception unit is configured to estimate the user's emotion and determine the priority of input based on the estimated emotion.
(Supplementary Note 12)The system according to Supplementary Note 1, wherein the reception unit is configured to prioritize the input of highly relevant information by considering the user's geographic location when entering photographs or situations of illness or injury.
(Supplementary Note 13)The system according to Supplementary Note 1, wherein the reception unit is configured to analyze the user's social media activity and input relevant information when entering photographs or situations of illness or injury.
(Supplementary Note 14)The system according to Supplementary Note 1, wherein the analysis unit is configured to estimate the user's emotion and adjust the accuracy of analysis based on the estimated emotion.
(Supplementary Note 15)The system according to Supplementary Note 1, wherein the analysis unit is configured to adjust the level of detail of analysis based on the importance of photographs or situations of illness or injury during analysis.
(Supplementary Note 16)The system according to Supplementary Note 1, wherein the analysis unit is configured to apply different analysis algorithms according to the category of illness or injury during analysis.
(Supplementary Note 17)The system according to Supplementary Note 1, wherein the analysis unit is configured to estimate the user's emotion and adjust the display method of the analysis result based on the estimated emotion.
(Supplementary Note 18)The system according to Supplementary Note 1, wherein the analysis unit is configured to determine the priority of analysis based on the occurrence time of the illness or injury during analysis.
(Supplementary Note 19)The system according to Supplementary Note 1, wherein the analysis unit is configured to determine the priority of analysis based on the occurrence time of the illness or injury during analysis.
(Supplementary Note 20)The system according to Supplementary Note 1, wherein the analysis unit is configured to refer to related literature on illness or injury during analysis to improve the accuracy of analysis.
(Supplementary Note 21)The system according to Supplementary Note 1, wherein the determination unit is configured to estimate the user's emotion and adjust the criteria for determining the necessity of medical consultation based on the estimated emotion.
(Supplementary Note 22)The system according to Supplementary Note 1, wherein the determination unit is configured to adjust the level of detail of determination based on the importance of the condition of the illness or injury during determination.
(Supplementary Note 23)The system according to Supplementary Note 1, wherein the determination unit is configured to apply different determination algorithms according to the category of illness or injury during determination.
(Supplementary Note 24)The system according to Supplementary Note 1, wherein the determination unit is configured to estimate the user's emotion and adjust the display method of the determination result based on the estimated emotion.
(Supplementary Note 25)The system according to Supplementary Note 1, wherein the determination unit is configured to determine the priority of determination based on the occurrence time of the illness or injury during determination.
(Supplementary Note 26)The system according to Supplementary Note 1, wherein the determination unit is configured to refer to related literature on illness or injury during determination to improve the accuracy of determination.
(Supplementary Note 27)The system according to Supplementary Note 1, wherein the request unit is configured to estimate the user's emotion and adjust the method of requesting an ambulance based on the estimated emotion.
(Supplementary Note 28)The system according to Supplementary Note 1, wherein the request unit is configured to adjust the level of detail of request based on the importance of the condition of the illness or injury during request.
(Supplementary Note 29)The system according to Supplementary Note 1, wherein the request unit is configured to apply different request algorithms according to the category of illness or injury during request.
(Supplementary Note 30)The system according to Supplementary Note 1, wherein the request unit is configured to estimate the user's emotion and adjust the display method of the request result based on the estimated emotion.
(Supplementary Note 31)The system according to Supplementary Note 1, wherein the request unit is configured to determine the priority of request based on the occurrence time of the illness or injury during request.
(Supplementary Note 32)The system according to Supplementary Note 1, wherein the request unit is configured to refer to related literature on illness or injury during request to improve the accuracy of request.
(Supplementary Note 33)The system according to Supplementary Note 1, wherein the provisional request unit is configured to estimate the user's emotion and adjust the method of provisional request to the hospital based on the estimated emotion.
(Supplementary Note 34)The system according to Supplementary Note 1, wherein the provisional request unit is configured to adjust the level of detail of provisional request based on the importance of the condition of the illness or injury during provisional request.
(Supplementary Note 35)The system according to Supplementary Note 1, wherein the provisional request unit is configured to apply different provisional request algorithms according to the category of illness or injury during provisional request.
(Supplementary Note 36)The system according to Supplementary Note 1, wherein the provisional request unit is configured to estimate the user's emotion and adjust the display method of the provisional request result based on the estimated emotion.
(Supplementary Note 37)The system according to Supplementary Note 1, wherein the provisional request unit is configured to determine the priority of provisional request based on the occurrence time of the illness or injury during provisional request.
(Supplementary Note 38)The system according to Supplementary Note 1, wherein the provisional request unit is configured to refer to related literature on illness or injury during provisional request to improve the accuracy of provisional request.

What is claimed is:

1. A system comprising:
circuitry configured to:
receive, from a client terminal via a packet-switched network, multimodal input data comprising at least one of image data, text data, or audio data;
analyze the multimodal input data by applying a neural network model to generate a severity score;
determine, based on the severity score, whether a service request condition is satisfied;
transmit, when the service request condition is satisfied, a service request to an external service system via the packet-switched network; and
transmit, when the service request has been transmitted, a provisional notification to a destination facility system via the packet-switched network, the provisional notification comprising at least one of an estimated arrival time or resource preparation data.

2. The system according to claim 1, wherein the multimodal input data comprises medical data of a user, the medical data comprising at least one of an injury photograph, a symptom description text, or a voice recording describing symptoms.

3. The system according to claim 2, wherein the circuitry is further configured to preprocess the image data by performing at least one of noise removal, resizing, or normalization, and converting the image data into a tensor format.

4. The system according to claim 1, wherein the neural network model comprises a convolutional neural network configured to analyze the image data and generate at least one of a segmentation map of an affected region or an abnormality probability score.

5. The system according to claim 1, wherein the neural network model comprises a natural language processing model configured to extract keywords from the text data and generate the severity score based on the extracted keywords.

6. The system according to claim 1, wherein the circuitry is further configured to convert the audio data into a spectrogram and extract acoustic features comprising mel-frequency cepstral coefficients.

7. The system according to claim 1, wherein the circuitry is further configured to estimate an emotion of a user based on at least one of the text data, the audio data, or the image data, and adjust a parameter of the neural network model based on the estimated emotion.

8. The system according to claim 7, wherein the circuitry is further configured to adjust a threshold for determining whether the service request condition is satisfied based on the estimated emotion, such that when the estimated emotion indicates anxiety, the threshold is set to a lower value.

9. The system according to claim 1, wherein the circuitry is further configured to calculate an importance score for the multimodal input data and adjust a level of detail of the analysis based on the importance score, such that for multimodal input data having a high importance score, the circuitry performs detailed analysis, and for multimodal input data having a low importance score, the circuitry performs simplified analysis.

10. The system according to claim 1, wherein the circuitry is further configured to determine a priority of the analysis based on an occurrence time associated with the multimodal input data, such that multimodal input data associated with a recent occurrence time is analyzed with a higher priority.

11. The system according to claim 1, wherein the circuitry is further configured to receive geographic location information of the user from the client terminal and select the destination facility system based on the geographic location information.

12. The system according to claim 1, wherein the provisional notification further comprises at least one of a condition summary, vital sign data, or a list of required resources.

13. The system according to claim 1, wherein the circuitry is further configured to estimate an emotion of a user and adjust a format of the provisional notification based on the estimated emotion, such that when the estimated emotion indicates urgency, the provisional notification is generated in a simplified format.

14. The system according to claim 1, wherein the circuitry is further configured to apply different analysis algorithms according to a category of the multimodal input data, such that for image data, the circuitry applies a convolutional neural network, and for text data, the circuitry applies a transformer-based language model.

15. The system according to claim 1, wherein the circuitry is further configured to retrieve related reference data from an external database based on the multimodal input data and adjust a parameter of the neural network model based on the retrieved reference data.

16. The system according to claim 1, wherein the circuitry is further configured to receive attribute information of the user from the client terminal, the attribute information comprising at least one of age, current health condition, or medical history, and adjust the analysis based on the attribute information.

17. The system according to claim 1, wherein the circuitry is further configured to receive social media activity data of the user from the client terminal, analyze the social media activity data using a natural language processing model to extract status information, and adjust the analysis based on the extracted status information.

18. A system comprising:
a communication interface configured to communicate with a client terminal, an external service system, and a destination facility system via a packet-switched network;
a processor;
a random-access memory;
a memory storing a neural network model and an emotion identification model; and
circuitry configured to:
receive, from the client terminal via the communication interface and the packet-switched network, multimodal input data comprising image data depicting an injury or illness, text data describing symptoms, and audio data of a user;
preprocess the image data by performing noise removal and normalization and converting the image data into a tensor format;
preprocess the audio data by performing spectrogram conversion and extracting mel-frequency cepstral coefficients;
analyze the multimodal input data by inputting the preprocessed image data into a convolutional neural network to generate at least one of a segmentation map or an abnormality score, inputting the text data into a natural language processing model to extract symptom keywords, and integrating features from the image data, the text data, and the audio data to generate a severity score;
estimate an emotion of the user by applying the emotion identification model to at least one of the text data, the audio data, or the image data;
determine, based on the severity score and a threshold adjusted according to the estimated emotion, whether a service request condition is satisfied;
transmit, when the service request condition is satisfied, a service request to the external service system via the communication interface and the packet-switched network, the service request comprising location information of the user; and
transmit, when the service request has been transmitted, a provisional notification to the destination facility system via the communication interface and the packet-switched network, the provisional notification comprising the severity score, an estimated arrival time, and a list of required resources.

19. The system according to claim 18, wherein the circuitry is further configured to select the destination facility system from a plurality of candidate facility systems based on the location information of the user and availability data received from the plurality of candidate facility systems.

20. A method performed by circuitry of a system, the method comprising:
receiving, from a client terminal via a packet-switched network, multimodal input data comprising at least one of image data, text data, or audio data;
analyzing the multimodal input data by applying a neural network model to generate a severity score;
determining, based on the severity score, whether a service request condition is satisfied;
transmitting, when the service request condition is satisfied, a service request to an external service system via the packet-switched network; and
transmitting, when the service request has been transmitted, a provisional notification to a destination facility system via the packet-switched network, the provisional notification comprising at least one of an estimated arrival time or resource preparation data.
